(19) United States
(12) Patent Application Publication
(10) Pub. No.: US 2026/0226423 A1
(43) Pub. Date: Aug. 6, 2026

(54) COMPOSITIONS AND METHODS FOR OBTAINING HUMAN INTESTINAL TISSUE AND RELATED USES THEREOF

(71) Applicant: The Regents of the University of Michigan, Ann Arbor, MI (US)

(72) Inventors: Madeline Eiken, Ann Arbor, MI (US); Charlie Childs, Ann Arbor, MI (US); Jason Spence, Ann Arbor, MI (US)

(21) Appl. No.: 19/576,555

(22) Filed: Mar. 24, 2026

Related U.S. Application Data
(62) Division of application No. 19/374,947, filed on Oct. 30, 2025.
(60) Provisional application No. 63/713,905, filed on Oct. 30, 2024.

(51) Int. Cl. C12N 5/071; C12N 5/00

(52) U.S. Cl. CPC C12N 5/0697; C12N 5/0018; C12N 2501/11; C12N 2501/155; C12N 2501/415; C12N 2502/081; C12N 2502/1347; C12N 2502/23; C12N 2506/02; C12N 2506/45; C12N 2510/00; C12N 2513/00

(57) ABSTRACT

The invention disclosed herein generally relates to methods and systems for converting stem cells into specific tissue(s) or organ(s) through directed differentiation. In particular, the invention disclosed herein relates to methods and systems for promoting human intestinal tissue (e.g., human intestinal organoid tissue) comprising epithelium, stroma, neurons, endothelial cells, and organized smooth muscle, and interstitial cells of Cajal (ICCs).

### CROSS REFERENCE TO RELATED APPLICATIONS

The present application is a divisional of U.S. patent application Ser. No. 19/374,947, filed Oct. 30, 2025, which claims priority to U.S. Provisional Application No. 63/713,905, filed Oct. 30, 2024, which are incorporated herein by reference in their entireties.

### STATEMENT REGARDING FEDERALLY SPONSORED RESEARCH OR DEVELOPMENT

This invention was made with government support under DK137806 awarded by the National Institutes of Health. The government has certain rights in the invention.

### FIELD OF THE INVENTION

The invention disclosed herein generally relates to methods and systems for converting stem cells into specific tissue(s) or organ(s) through directed differentiation. In particular, the invention disclosed herein relates to methods and systems for promoting human intestinal tissue (e.g., human intestinal organoid tissue) comprising epithelium, stroma, neurons, endothelial cells, organized smooth muscle, and interstitial cells of Cajal (ICCs).

### INTRODUCTION

Human intestinal organoids (HIOs) are three-dimensional structures derived from induced pluripotent stem cells (iPSCs) that model the human intestine¹ and are powerful tools for studying human development, physiology and pathophysiology²⁻⁵. Unlike patient-derived organoids (also known as enteroids), HIOs possess cells from multiple tissue lineages, including endoderm-derived epithelial cells and mesoderm-derived mesenchymal cells^{1,6}. HIOs are typically simple structures in vitro, lacking complex organization and important cell types crucial to intestinal function such as neurons, endothelial cells and organized smooth muscle. This simplicity renders the organoids incapable of recapitulating many functions of the intestine⁷. Several approaches have been developed to increase HIO complexity, including adding missing cell types individually or by co-culture, or transplantation into a murine host where they are vascularized by the host and undergo maturation, developing crypts and villi along with the muscularis mucosae and outer muscle layers, representing a miniature human intestine^{6,8,9}. Altering growth factors present during differentiation has also been used as a method to induce additional lineages¹⁰⁻¹⁴; however, a single, harmonized method to achieve coordinated differentiation of multiple cell lineages in the same organoid has not previously been reported.

What is needed in the art are methods and systems for accurately controlling the destination of the pluripotent stem cells in order to create the specific type of tissue or organ of desire.

The present invention addresses these needs.

### SUMMARY OF THE INVENTION

Experiments conducted during the course of developing embodiments for the present invention leveraged recent discoveries that the EGF-family member, EPIREGULIN (EREG), is an important stem cell niche factor in the developing human intestine¹⁵, and demonstrate that EREG promotes iPSC-derived HIOs containing epithelium, stroma, neurons, endothelial cells, organized smooth muscle, and interstitial cells of Cajal (ICCs), similar to the native human intestine. Once transplanted into a murine host, these cell populations were shown to further organize along a crypt-villus axis. It was shown that these HIOs can recapitulate important functions of the human intestine as they undergo rhythmic peristaltic contractions, suggesting a functional enteric nervous system (ENS). HIOs were further shown to develop vascular networks in vitro and in vivo, confirming blood vessel function.

Collectively, this enhanced HIO model system provides new opportunities in translational and regenerative medicine, providing a platform to interrogate gut motility disorders, neurogenic and myogenic dysfunction (e.g., Hirschsprung's disease, intestinal atresia and stenosis), drug-induced dysmotility and other complicated gut pathophysiologies that involve multiple tissue lineages. Such results represent a marked and unexpected improvement over prior discoveries which implemented a different EGF-family member (EGF) as the inclusion of EGF in iPSC-derived HIO culture media led to HIOs lacking organized and differentiated smooth muscle, endothelial cells and neurons (see, e.g., Spence J R, et al., Nature. 2011 Feb. 3; 470(7332):105-9. doi: 10.1038/nature09691; McCracken K W, Howell J C, Wells J M, Spence J R, Nat Protoc. 2011 Nov. 10; 6(12):1920-8. doi: 10.1038/nprot.2011.410 PMID: 22082986).

Accordingly, the present invention disclosed herein relates to methods and systems for converting stem cells into specific tissue(s) or organ(s) through directed differentiation. In particular, the invention disclosed herein relates to methods and systems for promoting human intestinal tissue (e.g., human intestinal organoid tissue) comprising epithelium, stroma, neurons, endothelial cells, organized smooth muscle, and ICCs.

In certain embodiments, the present invention provides a composition comprising human intestinal organoid tissue derived from human pluripotent stem cells, wherein the human intestinal organoid tissue derived from human pluripotent stem cells comprises epithelial cells, stromal cells (also known as fibroblast cells), neuronal cells, endothelial cells, organized smooth muscle, and ICCs. In some embodiments, the human pluripotent stem cells are embryonic stem cells. In some embodiments, the human pluripotent stem cells are induced pluripotent stem cells.

In some embodiments, the human intestinal organoid tissue is genetically engineered. In some embodiments, the smooth muscle expresses smooth muscle 22 (SM22) protein and/or the transgelin (TAGLN) RNA. the neuronal cells express TUBB3 and/or MAP2 at the protein and/or RNA level. In some embodiments, the endothelial cells express PECAM and/or CDH5 at the protein and/or RNA level. In some embodiments, the epithelial cells express CDX2 at the protein and/or RNA level.

In certain embodiments, the present invention provides a method, comprising culturing hindgut spheroid tissue in vitro, wherein the culturing results in differentiation of the hindgut spheroid tissue into tissue comprising human intestinal tissue comprising epithelial cells, stromal cells (also known as fibroblast cells), neuronal cells, endothelial cells, organized smooth muscle, and ICCs. In some embodiments, the human intestinal tissue is human intestinal organoid tissue.

In some embodiments, the differentiation of the hindgut spheroid tissue into tissue comprising human intestinal tissue is accomplished via either spontaneous generation or spontaneous aggregation.

In some embodiments, the culturing of the hindgut spheroid tissue in vitro comprises culturing the hindgut spheroid tissue with media comprising EREG. In some embodiments, the concentration of EREG in the medium is between about 10 ng/mL and about 100 ng/mL (e.g., 10 ng/mL, 25 ng/mL, 50 ng/mL, or 100 ng/mL). In some embodiments, the media comprising EREG further comprises a BMP signaling pathway inhibitor selected from, for example, Noggin, dorsomorphin, and LDN-193189. In some embodiments, the media comprising EREG further comprises a Wnt signaling pathway activator selected from, for example, R-Spondin1, R-Spondin-2, R-Spondin-3, and CHIR99021. In some embodiments, the media comprising EREG further comprises a BMP signaling pathway inhibitor (e.g., Noggin, dorsomorphin, LDN-193189) and/or a Wnt signaling pathway activator (e.g., R-Spondin1, R-Spondin-2, R-Spondin-3, CHIR99021).

In some embodiments, the media comprising epiregulin (EREG) and does not contain epidermal growth factor (EGF) or neuregulin 1 (NRG1). In some embodiments, the media comprising EREG further comprises one or both of Noggin and R-Spondin1 and does not contain other members from the EGF family (e.g., EGF, NRG1).

In some embodiments, the human intestinal tissue is maintained and expanded. In some embodiments, the human intestinal tissue is maintained and expanded with media comprising EREG. In some embodiments, the human intestinal tissue is maintained and expanded with media comprising EREG, but does not contain does not contain other members from the EGF family (e.g., EGF, NRG1). In some embodiments, the human intestinal tissue is maintained and expanded with media comprising EREG but does not contain EGF.

In some embodiments, the smooth muscle expresses SM22 protein/TAGLN RNA. In some embodiments, the neuronal cells express TUBB3 and/or MAP2 at the protein and/or RNA level. In some embodiments, the endothelial cells express PECAM and/or CDH5 at the protein and/or RNA level. In some embodiments, the epithelial cells express CDX2 at the protein and/or RNA level.

In some embodiments, the duration of differentiating the hindgut spheroid tissue into tissue comprising human intestinal organoid tissue is approximately three days (e.g., 10 hours, 11 hours, 12 hours, 13 hours, 14 hours, 15 hours, 16 hours, 17 hours, 20 hours, 22 hours, 23 hours, 24 hours, 25 hours, 26 hours, 27 hours, 28 hours, 30 hours, 33 hours, 34 hours, 35 hours, 36 hours, 37 hours, 38 hours, 39 hours, 40 hours, 42 hours, 44 hours, 45 hours, 46 hours, 47 hours, 48 hours, 49 hours, 50 hours, 51 hours, 52 hours, 57 hours, 58 hours, 59 hours, 60 hours, 61 hours, 62 hours, 63 hours, 64 hours, 65 hours, 66 hours, 75 hours, 100 hours, 125 hours, 150 hours, 175 hours, 200 hours, 210 hours, 225 hours, 238 hours, 239 hours, 240 hours, 241 hours, 242 hours, 243 hours, 250 hours) (e.g., 0.5 days, 1 day, 1.5 days, 2 days, 2.5 days, 2.75 days, 3 days, 3.1 days, 3.5 days, 4 days, 5 days, 5.5 days, up to 10 days).

In some embodiments, the hindgut spheroid tissue is obtained through culturing endoderm/mesoderm cells with a WNT signaling family activator (e.g., WNT3A, CHIR99021) and FGF4 for approximately six days (e.g., 30 hours, 33 hours, 34 hours, 35 hours, 36 hours, 37 hours, 38 hours, 39 hours, 40 hours, 42 hours, 44 hours, 45 hours, 46 hours, 47 hours, 48 hours, 49 hours, 50 hours, 51 hours, 52 hours, 57 hours, 58 hours, 59 hours, 60 hours, 61 hours, 62 hours, 63 hours, 64 hours, 65 hours, 66 hours, 75 hours, 100 hours, 125 hours, 150 hours, 175 hours, 200 hours, 210 hours, 225 hours, 238 hours, 239 hours, 240 hours, 241 hours, 242 hours, 243 hours, 250 hours, 300 hours, 350 hours, 400 hours, 450 hours, 500 hours, 550 hours, 600 hours) (e.g., 3 days, 4 days, 5 days, 6 days, 7 days, 8 days, 9 days, up to 20 days).

In some embodiments, the culturing results in spontaneous hindgut spheroid tissue formation. In some embodiments, the culturing results in hindgut spheroid tissue formation through cell-suspension re-aggregation.

In some embodiments, the endoderm/mesoderm cells are obtained through culturing induced pluripotent stem cells with Activin A for a period of approximately three days (e.g., 30 hours, 33 hours, 34 hours, 35 hours, 36 hours, 37 hours, 38 hours, 39 hours, 40 hours, 42 hours, 44 hours, 45 hours, 46 hours, 47 hours, 48 hours, 49 hours, 50 hours, 51 hours, 52 hours, 57 hours, 58 hours, 59 hours, 60 hours, 61 hours, 62 hours, 63 hours, 64 hours, 65 hours, 66 hours, 75 hours, 100 hours, 125 hours, 150 hours, 175 hours, 200 hours, 210 hours, 225 hours, 238 hours, 239 hours, 240 hours, 241 hours, 242 hours, 243 hours, 250 hours, 300 hours, 350 hours, 400 hours, 450 hours, 500 hours, 550 hours, 600 hours) (e.g., 1 day, 2 days, 3 days, 4 days, 5 days, up to 10 days).

In some embodiments, the pluripotent stem cells are human induced pluripotent stem cells or human embryonic stem cells.

In some embodiments, the culturing steps are conducted in vitro.

In certain embodiments, the present invention provides a composition comprising human intestinal organoid tissue comprising epithelial cells, stromal cells (also known as fibroblast cells), neuronal cells, endothelial cells, organized smooth muscle, and ICCs obtained through any of the methods described herein.

In certain embodiments, the present invention provides a method, comprising a) culturing human induced pluripotent stem cells (iPSCs) or human embryonic stem cells (hESCs) in a first culture medial comprising Activin A to produce endoderm/mesoderm cells; b) treating the endoderm/mesoderm cells with a WNT signaling family activator (e.g., WNT3A, CHIR99021) and FGF4 to produce hindgut spheroid tissue; and c) contacting the hindgut spheroid tissue with a media comprising EREG to produce human intestinal organoid tissue comprising epithelial cells, stromal cells (also known as fibroblast cells), neuronal cells, endothelial cells, organized smooth muscle, and ICCs.

In some embodiments, the media comprising EREG within step c further comprises one or both of a BMP signaling pathway inhibitor (e.g., Noggin, dorsomorphin, LDN-193189) and/or a Wnt signaling pathway activator (e.g., R-Spondin1, R-Spondin-2, R-Spondin-3, CHIR99021). In some embodiments, the media comprising EREG within step c further comprises a BMP signaling pathway inhibitor (e.g., Noggin, dorsomorphin, LDN-193189) and/or a Wnt signaling pathway activator (e.g., R-Spondin1, R-Spondin-2, R-Spondin-3, CHIR99021), but does not contain other members of the EGF family (e.g., EGF, NRG1). In some embodiments, the media comprising EREG within step c does not contain EGF.

In some embodiments, the produced human intestinal organoid tissue are maintained and expanded. In some embodiments, the produced human intestinal organoid tissue are maintained and expanded with media comprising EREG. In some embodiments, the produced human intestinal organoid tissue are maintained and expanded with media comprising EREG and not containing EGF or NRG1.

In certain embodiments, the present invention provides a kit comprising human intestinal organoid tissue comprising epithelial cells, stromal cells (also known as fibroblast cells), neuronal cells, endothelial cells, organized smooth muscle, and ICCs obtained through any of the methods described herein.

In certain embodiments, the present invention provides a composition comprising human intestinal tissue derived from human pluripotent stem cells, wherein the human intestinal tissue derived from human pluripotent stem cells comprises epithelial cells expressing CDX2, stromal cells (also known as fibroblast cells), neuronal cells, endothelial cells, and organized smooth muscle. In some embodiments, the human pluripotent stem cells are embryonic stem cells. In some embodiments, the human pluripotent stem cells are induced pluripotent stem cells.

In some embodiments, the human intestinal tissue derived from pluripotent stem cells is human intestinal organoid tissue derived from pluripotent stem cells.

In some embodiments, the smooth muscle expresses SM22 protein and/or TAGLN RNA. the neuronal cells express TUBB3 and/or MAP2 at the protein and/or RNA level. In some embodiments, the endothelial cells express PECAM and/or CDH5 at the protein and/or RNA level. In some embodiments, the epithelial cells express CDX2 at the protein and/or RNA level.

In certain embodiments, the present invention provides a method, comprising culturing hindgut spheroid tissue in vitro, wherein the culturing results in differentiation of the hindgut spheroid tissue into tissue comprising human intestinal tissue comprising epithelial cells expressing CDX2, stromal cells, neuronal cells, endothelial cells, organized smooth muscle, and ICCs.

In some embodiments, the human intestinal tissue is human intestinal organoid tissue.

In some embodiments, the differentiation of the hindgut spheroid tissue into tissue comprising human intestinal tissue is accomplished via either spontaneous generation or spontaneous aggregation.

In some embodiments, the culturing of the hindgut spheroid tissue in vitro comprises culturing the hindgut spheroid tissue with media comprising EREG. In some embodiments, the concentration of EREG in the medium is between about 10 ng/mL and about 100 ng/mL (e.g., 10 ng/mL, 25 ng/mL, 50 ng/mL, or 100 ng/mL). In some embodiments, the culturing of the hindgut spheroid tissue in vitro comprises culturing the hindgut spheroid tissue with media comprising EREG and does not contain EGF. In some embodiments, the media comprising EREG further comprises a BMP signaling pathway inhibitor selected from, for example, Noggin, dorsomorphin, and LDN-193189. In some embodiments, the media comprising EREG further comprises a Wnt signaling pathway activator selected from, for example, R-Spondin1, R-Spondin-2, R-Spondin-3, and CHIR99021. In some embodiments, the media comprising EREG further comprises a BMP signaling pathway inhibitor (e.g., Noggin, dorsomorphin, LDN-193189) and/or a Wnt signaling pathway activator (e.g., R-Spondin1, R-Spondin-2, R-Spondin-3, CHIR99021). In some embodiments, the media comprising EREG further comprises one or both of Noggin and R-Spondin1. In some embodiments, the media comprising EREG further comprises one or both of Noggin and R-Spondin1 but does not contain other members of the EGF family (e.g., EGF, NRG1).

In some embodiments, the human intestinal tissue is maintained and expanded. In some embodiments, the human intestinal tissue is maintained and expanded with media comprising EREG. In some embodiments, the human intestinal tissue is maintained and expanded with media comprising EREG, but does not contain does not contain other members from the EGF family (e.g., EGF, NRG1). In some embodiments, the human intestinal tissue is maintained and expanded with media comprising EREG but does not contain EGF.

In some embodiments, the smooth muscle expresses SM22 protein and/or TAGLN RNA. In some embodiments, the neuronal cells express TUBB3 and/or MAP2 at the protein and/or RNA level. In some embodiments, the endothelial cells express PECAM and/or CDH5 at the protein and/or RNA level. In some embodiments, the epithelial cells express CDX2 at the protein and/or RNA level.

In some embodiments, the duration of differentiating the hindgut spheroid tissue into tissue comprising human intestinal organoid tissue is approximately three days (e.g., 10 hours, 11 hours, 12 hours, 13 hours, 14 hours, 15 hours, 16 hours, 17 hours, 20 hours, 22 hours, 23 hours, 24 hours, 25 hours, 26 hours, 27 hours, 28 hours, 30 hours, 33 hours, 34 hours, 35 hours, 36 hours, 37 hours, 38 hours, 39 hours, 40 hours, 42 hours, 44 hours, 45 hours, 46 hours, 47 hours, 48 hours, 49 hours, 50 hours, 51 hours, 52 hours, 57 hours, 58 hours, 59 hours, 60 hours, 61 hours, 62 hours, 63 hours, 64 hours, 65 hours, 66 hours, 75 hours, 100 hours, 125 hours, 150 hours, 175 hours, 200 hours, 210 hours, 225 hours, 238 hours, 239 hours, 240 hours, 241 hours, 242 hours, 243 hours, 250 hours) (e.g., 0.5 days, 1 day, 1.5 days, 2 days, 2.5 days, 2.75 days, 3 days, 3.1 days, 3.5 days, 4 days, 5 days, 5.5 days, up to 10 days).

In some embodiments, the hindgut spheroid tissue is obtained through culturing endoderm/mesoderm cells with a WNT signaling family activator (e.g., WNT3A, CHIR99021) and FGF4 for approximately six days (e.g., 30 hours, 33 hours, 34 hours, 35 hours, 36 hours, 37 hours, 38 hours, 39 hours, 40 hours, 42 hours, 44 hours, 45 hours, 46 hours, 47 hours, 48 hours, 49 hours, 50 hours, 51 hours, 52 hours, 57 hours, 58 hours, 59 hours, 60 hours, 61 hours, 62 hours, 63 hours, 64 hours, 65 hours, 66 hours, 75 hours, 100 hours, 125 hours, 150 hours, 175 hours, 200 hours, 210 hours, 225 hours, 238 hours, 239 hours, 240 hours, 241 hours, 242 hours, 243 hours, 250 hours, 300 hours, 350 hours, 400 hours, 450 hours, 500 hours, 550 hours, 600 hours) (e.g., 3 days, 4 days, 5 days, 6 days, 7 days, 8 days, 9 days, up to 20 days).

In some embodiments, the culturing results in spontaneous hindgut spheroid tissue formation.

In some embodiments, the culturing results in hindgut spheroid tissue formation through cell-suspension re-aggregation.

In some embodiments, the endoderm/mesoderm cells are obtained through culturing induced pluripotent stem cells with Activin A for a period of approximately three days (e.g., 10 hours, 11 hours, 12 hours, 13 hours, 14 hours, 15 hours, 16 hours, 17 hours, 20 hours, 22 hours, 23 hours, 24 hours, 25 hours, 26 hours, 27 hours, 28 hours, 30 hours, 33 hours, 34 hours, 35 hours, 36 hours, 37 hours, 38 hours, 39 hours, 40 hours, 42 hours, 44 hours, 45 hours, 46 hours, 47 hours, 48 hours, 49 hours, 50 hours, 51 hours, 52 hours, 57 hours, 58 hours, 59 hours, 60 hours, 61 hours, 62 hours, 63 hours, 64 hours, 65 hours, 66 hours, 75 hours, 100 hours, 125 hours, 150 hours, 175 hours, 200 hours, 210 hours, 225 hours, 238 hours, 239 hours, 240 hours, 241 hours, 242 hours, 243 hours, 250 hours) (e.g., 1 day, 2 days, 3 days, 4 days, 5 days, up to 10 days).

In some embodiments, the pluripotent stem cells are human induced pluripotent stem cells or human embryonic stem cells.

In some embodiments, the culturing steps are conducted in vitro.

In certain embodiments, the present invention provides a composition comprising human intestinal organoid tissue comprising epithelial cells, stromal cells (also known as fibroblast cells), neuronal cells, endothelial cells expressing CDX2, organized smooth muscle, and ICCs obtained through any of the methods described herein.

In certain embodiments, the present invention provides a method, comprising a) culturing human induced pluripotent stem cells (iPSCs) or human embryonic stem cells (hESCs) in a first culture medial comprising Activin-A to produce endoderm/mesoderm cells; b) treating the endoderm/mesoderm cells with a WNT signaling family activator (e.g., WNT3A, CHIR99021) and FGF4 to produce hindgut spheroid tissue; and c) contacting the hindgut spheroid tissue with a media comprising EREG to produce human intestinal organoid tissue comprising epithelial cells, stromal cells (also known as fibroblast cells), neuronal cells, endothelial cells expressing CDX2, organized smooth muscle, and ICCs.

In some embodiments, the media comprising EREG within step c further comprises a BMP signaling pathway inhibitor (e.g., Noggin, dorsomorphin, LDN-193189) and/or a Wnt signaling pathway activator (e.g., R-Spondin1, R-Spondin-2, R-Spondin-3, CHIR99021). In some embodiments, the media comprising EREG within step c further comprises one or both of Noggin and R-Spondin1 but does not contain other members of the EGF family (e.g., EGF, NRG1). In some embodiments, the media comprising EREG within step c does not contain EGF or NRG1.

In some embodiments, the human intestinal tissue is maintained and expanded. In some embodiments, the human intestinal tissue is maintained and expanded with media comprising EREG. In some embodiments, the human intestinal tissue is maintained and expanded with media comprising EREG, but does not contain does not contain other members from the EGF family (e.g., EGF, NRG1). In some embodiments, the human intestinal tissue is maintained and expanded with media comprising EREG but does not contain EGF.

In certain embodiments, the present invention provides a kit comprising human intestinal organoid tissue comprising epithelial cells, stromal cells (also known as fibroblast cells), neuronal cells, endothelial cells, organized smooth muscle, and ICCs obtained through any of the methods described herein.

In certain embodiments, the present invention provides a composition comprising human intestinal organoid tissue derived from human pluripotent stem cells, wherein the organoid tissue comprises epithelial cells, stromal (fibroblast) cells, neuronal cells, endothelial cells, organized smooth muscle, and ICCs, and wherein the organoid tissue is produced by culturing hindgut spheroid tissue in a medium comprising EREG.

In some embodiments, the medium further comprises a BMP signaling pathway inhibitor (e.g., Noggin, dorsomorphin, LDN-193189) and/or a Wnt signaling pathway activator (e.g., R-Spondin1, R-Spondin-2, R-Spondin-3, CHIR99021).

In some embodiments, the medium does not contain EGF or NRG1.

In some embodiments, the human pluripotent stem cells are embryonic stem cells.

In some embodiments, the human pluripotent stem cells are induced pluripotent stem cells.

In some embodiments, the smooth muscle expresses smooth muscle 22 (SM22) protein and/or transgelin (TAGLN); and/or the neuronal cells express TUBB3 and/or MAP2 at the protein and/or RNA level; and/or the epithelial cells express CDX2 at the protein and/or RNA level.

In certain embodiments, the present invention provides a method for producing human intestinal organoid tissue, comprising culturing hindgut spheroid tissue in vitro in a medium comprising EREG under conditions effective to differentiate the hindgut spheroid tissue into human intestinal organoid tissue comprising epithelial cells, stromal cells, neuronal cells, endothelial cells, organized smooth-muscle cells, and ICCs.

In some embodiments, the EREG is present in the medium at a concentration between about 10 ng/mL and about 100 ng/mL.

In some embodiments, the medium further comprises a BMP signaling pathway inhibitor (e.g., Noggin, dorsomorphin, LDN-193189) and/or a Wnt signaling pathway activator (e.g., R-Spondin1, R-Spondin-2, R-Spondin-3, CHIR99021).

In some embodiments, the medium does not contain EGF or NRG1.

In some embodiments, the human intestinal organoid tissue is maintained or expanded in a medium comprising EREG.

### BRIEF DESCRIPTION OF DRAWINGS

The patent or application file contains at least one drawing executed in color. For purposes of publication, the figures have been converted to grayscale.

FIG. 1A-E: EREG is expressed in the developing human intestine's crypts and smooth muscle bands across developmental time. A) UMAP visualization of Louvain clustering by cell type of human fetal intestine (51,790 cells, n=13 biological samples) for major tissue classifications. B) UMAP visualization of clustering by age of human fetal intestine (n=13 biological samples. 59-, 72-, 80-, 85-, 101-, 127-, and 132-days post conception). C) Dot plot of fetal dataset highlighting expression of canonical lineage genes used for cluster annotation by tissue type. D) Dot plot of EREG expression in epithelial clusters (marked by CDH1) and smooth muscle clusters (marked by ACTA2 and TAGLN). E) Co-FISH/IF staining for EREG (pink), DAPI (grey), ECAD (blue), and SM22 (green) in the developing human intestine at select timepoints across developmental time.

FIG. 2A-D: EREG-HIOs grown in vitro spontaneously and simultaneously pattern endothelium, smooth muscle, and neural components. A) Schematic of HIO directed differentiation using standard EGF conditions (grey) and experimental EREG conditions (pink). B) UMAP visualization of snRNA-seq from 28-day in vitro grown EREG-HIOs in 10 ng/mL of EREG (n=1 sequencing run of over 20 combined HIOs). C) Dot plot visualization for expression of canonical markers of neurons (S100B, PLP1, STMN2, ELAVL4), endothelial cells (CDH5, KDR, ECSCR, CLDN5), mesenchyme (COL1A1, COL1A2, DCN), smooth muscle (ACTA2, TAGLN, ACTG2, MYLK), epithelium (EPCAM, CDH1, CDX2, CLDN4) immune cells (PTPRC, ARHGDIB, CORO1A) and proliferative cells (MKI67, TOP2A), in EREG-grown (10 ng/mL) HIOs. Top panels: representative whole mount immunofluorescence (IF) staining of 10 ng/mL EGF- or EREG-grown HIOs for the presence of smooth muscle (SM22; green), endothelial cells (PECAM; pink), and neurons (TUBB3; blue). Inlays show IF staining of mesenchyme (VIM; pink) and epithelium (ECAD; green). Bottom panels: representative IF staining on 2D sections of EREG-grown (10 ng/mL) and EGF-grown (10 ng/mlL) HIOs for the presence of epithelium (ECAD; green), smooth muscle (SM22; blue), endothelial cells (PECAM; yellow), and neurons (MAP2; pink). All Scale bars=100 μm.

FIG. 3A-F: Measurements of forming efficiency, area, and shape of control EGF-grown HIOs compared to EREG-grown HIOs. A) Organoid forming efficiency assay results (n=3 biological replicates with n=3 technical replicates quantified for three different cell lines). Statistical significance was determined using an unpaired Welch's t test (**−P=0.0097, ns−P=>0.05). B-F) Morphologic quantifications including area, solidity, aspect ratio, circularity, and roundness of HIOs grown in varying doses of EGF or EREG. HIOs were derived from three separate cell lines and grown for 30 days. Three HIOs per condition were measured and the ImageJ analysis software was used to calculate these measurements. See methods section for further explanation on calculations. Statistical significance was determined using an unpaired Welch's t test (ns−P=>0.05).

FIG. 4A-H: Gene expression analysis of EREG-grown HIOs show presence of smooth muscle, neural cells, and endothelial cells not seen in control EGF-grown HIOs. A) UMAP visualization of Louvain clustering by cell type of all HIO samples sequenced (100 ng/mL EREG, 10 ng/mL EREG, 1 ng/mL EREG, and 100 ng/mL EGF). B) UMAP visualization of clustering by sample type (100 ng/mL EREG blue, 10 ng/mL EREG purple, 1 ng/mL EREG green, and 100 ng/mL EGF grey). Inlays break out each sample (red) individually against rest of dataset (grey). C) Dot plot of combined HIO dataset highlighting expression of canonical lineage genes used for cluster annotation. D) Bar charts showing the cell type abundance (% of total cells) within each cluster for each sample sequenced. Colors are consistent with the cell type annotation in panel A. E) UMAP visualization of Louvain clustering by cell type of 1 ng/mL EREG and accompanying dot plot of expression of canonical lineage genes used for cluster annotation. F) UMAP visualization of Louvain clustering by cell type of 10 ng/mL EREG and accompanying dot plot of expression of canonical lineage genes used for cluster annotation. G) Dot plot of individual HIO dataset highlighting expression of major ENS neuronal cell types seen in the developing human intestine. Enteric ganglion cells (TUBB3, SYN1), submucosal secretomotor (VIP), enteric glial cells (S100b—glial network; SOX10—EGC nuclei), ICC's (ANO1), cholinergic neurons (CHAT) and Schwann cells (MPZ, PLP1). H) Bar charts showing gene expression of smooth muscle (TAGLN), endothelial cells (CDH5, VEGF) and Neurons (RET, TUBB3) for 100 ng/mL, 10 ng/mL, and 1 ng/mL EREG and matched EGF HIOs. Data points shown are the average of triplicates completed in 3 different passages (batches) for 3 different cell lines. Statistical significance was determined using an unpaired Welch's t test to the standard 100 ng/mL EGF condition (ns−P=>0.05).

FIG. 5A-F: EREG-grown HIOs further mature and spatially organize after transplantation into murine kidney capsule. A) Schematic timeline of HIO transplantation experiment. B) Representative IF staining of human fetal intestine (left panel; 127 days post conception), EGF-grown tHIO (middle panel; 12 weeks), and EREG-grown tHIO (right panel; 12 weeks) stained for the presence of smooth muscle (SM22; green), epithelium (ECAD; blue), and neurons (TUBB3; pink) in top panels. Bottom panels show stains for the presence of smooth muscle (SM22; green), epithelium (ECAD; blue), and endothelial cells (PECAM; pink). All scale bars=50 μm. C) UMAP visualization of snRNA-seq of 12-week in vivo grown tHIOs in 10 ng/mL of EREG (n=1 sequencing run of one tHIO). D) Dot plot visualization for expression of canonical markers of neurons (S1OOB, PLP1, STMN2, ELAVL4), endothelial cells (CDH5, KDR, ECSCR, CLDN5), mesenchyme (VIM, COL1A1, COL1A2, DCN), smooth muscle (ACTA2, TAGLN, ACTG2, MYLK), epithelium (EPCAM, CDH1, CDX2, CLDN4) immune cells (PTPRC, HLA-DRA, ARHGDIB, CORO1A) and proliferative cells (MKI67, TOP2A).E) Left: UMAP visualization of human fetal intestinal data set from FIG. 1 recolored for cell type lineages: mesenchymal cells(red), epithelial cells (blue), immune cells (yellow), smooth muscle cells (green), endothelial cells (pink), and neuronal cells (purple). Right: UMAP visualization of label transfer results with reference human fetal intestinal dataset in grey and tHIO dataset in red. F) Violin plot quantification of cell type scoring for each tissue type in reference intestinal dataset.

FIG. 6A-B: EREG-grown tHIO pattern two off target epithelial clusters. A) Bar plot of clusters 5 and 7 showing predicted organ identity using the scoreHIO R package. Clustered mapped somewhere between gastric epithelium and intestinal epithelium. B) Dot plot of human fetal dataset highlighting expression of canonical lineage genes used for cluster annotation in label transfer and SingleR analysis.

FIG. 7A-H: Assessment of EREG-grown tHIOs for neuromuscular units and native functionality. A) Experimental schematic for transplanting in vitro grown HIOs under the kidney capsule of a murine host and testing muscular and ENS function in an organ bath measured with isometric-force transducers post-transplant. B) Isometric force contractions in tissues isolated from n=4 different EGF-grown tHIOs (grey) and four different EREG-grown tHIOs (pink) after an equilibrium period with no exogenous contractile triggers. C) Representative IF staining of human fetal intestine (Left; 127 days post conception), EGF-grown tHIO (Middle; 12-weeks), EREG-grown tHIO (Right; 12-weeks) stained for the presence of epithelium (ECAD; blue), general neurons (TUBB3; pink) and ICC's (c-KIT; green). All scale bars=50 μm. D) Activation of muscarinic receptor-induced contractions in tissues isolated from n=2 EGF-grown tHIOs (grey) and n=2 EREG-grown tHIOs (pink) using increasing doses of bethanechol. E) Inhibition of the muscarinic receptor with scopolamine induced muscle relaxation. Graphs show calculated maximum and minimum tissue tension from n=2 EGF-grown tHIOs (grey) and n=2 EREG-grown tHIOs (pink). F) Functional test of ENS inhibition using the neurotoxin tetrodotoxin (TTX). Addition of TTX lowers ENS activation in the presence of DMPP stimulation. Graphed is the change in AUC following a control DMPP stimulation measured after stimulation, followed by TTX treatment and a final DMPP stimulation in EREG-grown tHIOs. G) Functional test of specific ENS neuronal types (nitrergic and cholinergic) in muscle contractions. Inhibition using the nitrergic inhibitor L-NAME and the cholinergic inhibitor atropine. Graphed is the change in AUC following a control DMPP stimulation measured after stimulation, followed by L-NAME treatment, another DMPP stimulation, followed by Atropine treatment, and a final DMPP stimulation in EREG-grown tHIOs.

FIG. 8A-H: EREG-grown HIOs pattern blood vessels that are functional both in vitro and in vivo. A) Schematic of workflow for whole mount imaging in vitro EREG-grown HIOs. B) Representative IF staining of n=4 different EREG-grown (10 ng/mL) HIOs for the presence of endothelial cells (PECAM; red), and DAPI (grey). All scale bars=100 μm. C) Schematic of workflow for in vitro EREG-grown HIO functionality test using RVEC microfluidic device. D) Representative IF staining of the control RVEC only microfluidic device lane (top) and the RVEC+EREG-grown (10 ng/mL) HIOs lane. RVECs (green), mCherry+HIOs (yellow), lectin dye flown through system (red) and PECAM dye flown through system (blue). Overlap (purple) in HIOs are areas where RVECs connected with endogenous endothelial cells and lectin flow moved through vessels. Control scale bars=100 μm and RVEC+HIO image scale bars=20 μm. E) Schematic of workflow for in vivo EREG-grown tHIO functionality test for connection with host vasculature. F) Representative whole mount IF staining of EGF-grown (10 ng/mL) tHIOs for the presence of human endothelial cells (hsPECAM; red), HIO mCherry tag (white), lectin dye administered through tail vein injection (yellow) and DAPI (blue). All scale bars=100 μm. G) Representative whole mount IF staining of EREG-grown (10 ng/mL) tHIOs for the presence of human endothelial cells (hsPECAM; red), HIO mCherry tag (white), lectin dye administered through tail vein injection (yellow) and DAPI (blue). All scale bars=100 μm. H) Quantification of flow cytometry analysis to quantify the percentage of hsPECAM⁺/Lectin⁺ cells. Three 12-week-old tHIOs per condition were pooled per condition to ensure enough material for experiment.

FIG. 9A-D: EREG-grown tHIOs feature human blood vessels that can connect to their murine host. A) IF staining of human fetal intestine (Left; 127-days post conception) and E13.5 mouse intestine to test for human specificity of hsPECAM (pink) antibody compared to a pan-species VE-CAD antibody (yellow) and smooth muscle SM22 (blue). All Scale bars=50 μm. B) IF staining of EREG-grown tHIO (Left) and EGF-grown tHIO (Right) with stains for DAPI (grey), autofluorescent red blood cells in the 488-laser channel (red), and human specific PECAM antibody (yellow). All scale bars=50 μm. C) IF staining of other areas of EREG-grown tHIO with stains for DAPI (grey), autofluorescent red blood cells in the 488-laser channel (red), and human specific PECAM antibody (yellow). All Scale bars=50 μm. D) IF staining of EREG-grown tHIO vessels (co-stain of hsPECAM in pink with pan-species VE-CAD in yellow) connecting to a mouse blood vessel (stained for only pan-species VE-CAD yellow) with smooth muscle SM22 (blue). All Scale bars=50 μm.

FIG. 10A-B: Flow plots for human specific lectin vessel quantification. A) Flow cytometric plots of batch-matched 12-week control EGF-grown HIOs for mCherry tag, human specific CD144/VE-CAD, and Lectin. Flow cytometric analysis required multiple tHIOs to be pooled from the same batch to ensure enough cells for experiment. B) Flow cytometric plots of batch-matched 12-week EREG-grown HIOs for mCherry tag, human specific CD144/VE-CAD, and Lectin. Flow cytometric analysis required multiple tHIOs to be pooled from the same batch to ensure enough cells for experiment.

FIG. 11A-B demonstrates that human intestinal organoids grown in EREG consistently show higher expression of genes associated with these cell types compared to their EGF counterparts. A) Experimental design schematic: Spheroids were collected from directed differentiation protocol and immediately placed in either EREG+NOGGIN+RSPO1 (E*NR) or EGF+NOGGIN+RSPO1 (ENR) for 3 days. After this initial step, the experiment was split into 3 conditions: 1) E*NR-minigut/ENR-minigut 2) E*NREREG-minigut/ENR-EGF-minigut 3) E*NR-EREG/ENR-EGF. The first condition removed EGF and EREG after three days and grew the spheroids in a basal media until they were 21 days old. The second condition removed NOGGIN+RSPO1 after 3 days, allowed the spheroids to grow in EREG or EGF only for an additional 4 days, and then removed EREG or EGF completely and grew them in just basal media until they were 21 days old. The third condition removed NOGGIN+RSPO1 after 3 days, then grew organoids in EREG or EGF only until they were 21 days old (this condition is the standard way HIOs are grown). Organoids were collected for qPCR at 21 days. B) qPCR results from organoids collected in (A) for expression of genes associated with smooth muscle (TAGLN), endothelial cells (CDH5, PECAM), neurons (TUBB3, MAP2), and intestinal epithelial marker (CDX2). Organoids grown in EREG for any amount of time were shown to have more expression of these genes than their counterparts grown in EGF suggest EREG is driving the creation of these cell types uniquely from EGF.

### DEFINITIONS

As used in the description of the invention and the claims, the singular forms “a,” “an,” and “the” are intended to include the plural forms as well unless the context indicates otherwise.

As used herein, the term “embryonic stem cells (ESCs),” also commonly abbreviated as ES cells, refers to cells that are pluripotent and derived from the inner cell mass of the blastocyst, an early-stage embryo. For purpose of the present invention, the term “ESCs” is used broadly sometimes to encompass the embryonic germ cells as well.

As used herein, the term “pluripotent stem cells (PSCs),” also commonly known as PS cells, encompasses any cells that can differentiate into nearly all cells, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of totipotent cells or obtained through induction of a non-pluripotent cell, such as an adult somatic cell, by forcing the expression of certain genes.

As used herein, the term “induced pluripotent stem cells (iPSCs),” also commonly abbreviated as iPS cells, refers to a type of pluripotent stem cells artificially derived from a normally non-pluripotent cell, such as an adult somatic cell, by inducing a “forced” expression of certain genes.

The term “I-iPSCs” refers to human induced pluripotent stem cells with similar phenotypical and genotypical characteristics as human embryonic stem cells (hESCs) or human pluripotent stem cells (HPSCs). HPSCs have self-renewing capabilities similar to hESCs and can undergo three germ layers, producing all the germ layer cells with appropriate growth factors such as endodermal lineage-derived intestinal cells or organoids.

As used herein, the term “precursor cell” encompasses any cells that can be used in methods described herein, through which one or more precursor cells acquire the ability to renew itself or differentiate into one or more specialized cell types. In some embodiments, a precursor cell is pluripotent or has the capacity to becoming pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multipotent stem cell; an oligopotent stem cells and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment.

In developmental biology, cellular differentiation is the process by which a less specialized cell becomes a more specialized cell type. As used herein, the term “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

As used herein, the term “cellular constituents” are individual genes, proteins, mRNA expressing genes, and/or any other variable cellular component or protein activities such as the degree of protein modification (e.g., phosphorylation), for example, that is typically measured in biological experiments (e.g., by microarray or immunohistochemistry) by those skilled in the art. Significant discoveries relating to the complex networks of biochemical processes underlying living systems, common human diseases, and gene discovery and structure determination can now be attributed to the application of cellular constituent abundance data as part of the research process. Cellular constituent abundance data can help to identify biomarkers, discriminate disease subtypes and identify mechanisms of toxicity.

As used herein, the term “organoid” is used to mean a 3-dimensional growth of mammalian cells in culture that retains characteristics of the tissue in vivo, e.g. prolonged tissue expansion with proliferation, multilineage differentiation, recapitulation of cellular and tissue ultrastructure, etc.

As used herein, “differentiate” or “differentiated” are used to refer to the process and conditions by which immature (unspecialized) cells acquire characteristics, becoming mature (specialized) cells, thereby acquiring particular form and function. Stem cells (unspecialized) are often exposed to varying conditions (e.g., growth factors and morphogenic factors) to induce specified lineage commitment, or differentiation, of said stem cells. The process by which an unspecialized (“uncommitted”) or less specialized cell acquires the features of a specialized cell such as, for example, a blood cell or a muscle cell. A differentiated or differentiation-induced cell has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or a subset of cell types, and cannot, under normal circumstances, differentiate into a different cell type or revert to a less differentiated cell type.

As used herein, the terms “genetically modified” or “engineered” cell as used herein refers to a cell into which an exogenous nucleic acid has been introduced by a process involving the hand of man (or a descendant of such a cell that has inherited at least a portion of the nucleic acid). The nucleic acid may for example contain a sequence that is exogenous to the cell, it may contain native sequences (i.e., sequences naturally found in the cells) but in a non-naturally occurring arrangement (e.g., a coding region linked to a promoter from a different gene), or altered versions of native sequences, etc. The process of transferring the nucleic acid into the cell can be achieved by any suitable technique. Suitable techniques include calcium phosphate or lipid-mediated transfection, electroporation, and transduction or infection using a viral vector. In some embodiments the polynucleotide or a portion thereof is integrated into the genome of the cell. The nucleic acid may have subsequently been removed or excised from the genome, provided that such removal or excision results in a detectable alteration in the cell relative to an unmodified but otherwise equivalent cell. It should be appreciated that the term genetically modified is intended to include the introduction of a modified RNA directly into a cell (e.g., a synthetic, modified RNA). Such synthetic modified RNAs include modifications to prevent rapid degradation by endo- and exo-nucleases and to avoid or reduce the cell's innate immune or interferon response to the RNA. Modifications include, but are not limited to, (a) end modifications, e.g., 5′ end modifications (phosphorylation dephosphorylation, conjugation, inverted linkages, etc.) or 3′ end modifications (conjugation, DNA nucleotides, inverted linkages, etc.); (b) base modifications, e.g., replacement with modified bases, stabilizing bases, destabilizing bases, bases that base pair with an expanded repertoire of partners, or conjugated bases; (c) sugar modifications (e.g., at the 2′ position or 4′ position) or replacement of the sugar; and (d) internucleoside linkage modifications, including modification or replacement of the phosphodiester linkages. To the extent that such modifications interfere with translation, the modification is not suitable for the methods and compositions described herein.

As used herein, the term “interstitial cells of Cajal” or “ICCs” refers to specialized pacemaker cells located within the gastrointestinal tract that generate rhythmic electrical slow waves and coordinate smooth muscle contractions.

### DETAILED DESCRIPTION OF THE INVENTION

Human intestinal organoids (HIOs) derived from human pluripotent stem cells co-differentiate both epithelial and mesenchymal lineages in vitro but lack important cell types such as neurons, endothelial cells, and smooth muscle, which limits translational potential and the ability to model complex physiology and pathophysiology.

Experiments described herein (see, Examples I-VI) demonstrate that the intestinal stem cell niche factor, EPIREGULIN (EREG) enhances HIO differentiation with epithelium, mesenchyme, enteric neuroglial populations, endothelial cells, and organized smooth muscle in a single differentiation, without the need for co-culture. When transplanted into a murine host, were shown to HIOs mature and demonstrate enteric nervous system function, undergoing peristaltic-like contractions indicative of a functional neuromuscular unit. HIOs were also shown to form functional vasculature, demonstrated in vitro using microfluidic devices to introduce vascular-like flow, and in vivo following transplantation, where HIO endothelial cells anastomose with host vasculature. These enhanced HIOs represent a transformative tool for translational research in the human gut, may be used to interrogate complex gut-related diseases, and may be used for testing therapeutic interventions with high fidelity to human pathophysiology.

Accordingly, the present invention disclosed herein relates to methods and systems for converting stem cells into specific tissue(s) or organ(s) through directed differentiation. In particular, the invention disclosed herein relates to methods and systems for promoting human intestinal tissue (e.g.., human intestinal organoid tissue) comprising epithelium, stroma, neurons, endothelial cells, and organized smooth muscle, and interstitial cells of Cajal (ICCs).

An important step for any of the compositions and methods of the present invention is to obtain stem cells that are pluripotent or can be induced to become pluripotent. In some embodiments, pluripotent stem cells are derived from embryonic stem cells, which are in turn derived from totipotent cells of the early mammalian embryo and are capable of unlimited, undifferentiated proliferation in vitro. Embryonic stem cells are pluripotent stem cells derived from the inner cell mass of the blastocyst, an early-stage embryo. Methods for deriving embryonic stem cells from blastocytes are well known in the art. For example, three cell lines (H1, H13, and H14) have a normal XY karyotype, and two cell lines (H7 and H9) have a normal XX karyotype.

Additional stem cells that can be used in embodiments in accordance with the present invention include but are not limited to those provided by or described in the database hosted by the National Stem Cell Bank (NSCB), Human Embryonic Stem Cell Research Center at the University of California, San Francisco (UCSF); WISC cell Bank at the Wi Cell Research Institute; the University of Wisconsin Stem Cell and Regenerative Medicine Center (UW-SCRMC); Novocell, Inc. (San Diego, Calif.); Cellartis AB (Goteborg, Sweden); ES Cell International Pte Ltd (Singapore); Technion at the Israel Institute of Technology (Haifa, Israel); and the Stem Cell Database hosted by Princeton University and the University of Pennsylvania. Indeed, embryonic stem cells that can be used in embodiments in accordance with the present invention include but are not limited to SA01 (SA001); SA02 (SA002); ESO1 (HES-1); ES02 (HES-2); ES03 (HES-3); ES04 (HES-4); ES05 (HES-5); ES06 (HES-6); BGO1 (BGN-01); BG02 (BGN-02); BG03 (BGN-03); TE03 (13); TE04 (14); TE06 (16); UCO1 (HSF1); UC06 (HSF6); WA01 (H1); WA07 (H7); WA09 (H9); WA13 (H13); WA14 (H14).

In some embodiments, the stem cells are further modified to incorporate additional properties. Exemplary modified cell lines include but not limited to H1 OCT4-EGFP; H9 Cre-LoxP; H9 hNanog-pGZ; H9 hOct4-pGZ; H9 in GFPhES; and H9 Syn-GFP.

More details on embryonic stem cells can be found in, for example, Thomson et al., 1998, Science 282 (5391):1145-1147; Andrews et al., 2005, Biochem Soc Trans 33:1526-1530; Martin 1980, Science 209 (4458):768-776; Evans and Kaufman, 1981, Nature 292(5819): 154-156; Klimanskaya et al., 2005, Lancet 365 (9471): 1636-1641).

Alternatively, pluripotent stem cells can be derived from embryonic germ cells (EGCs), which are the cells that give rise to the gametes of organisms that reproduce sexually. EGCs are derived from primordial germ cells found in the gonadal ridge of a late embryo, have many of the properties of embryonic stem cells. The primordial germ cells in an embryo develop into stem cells that in an adult generate the reproductive gametes (sperm or eggs). In mice and humans it is possible to grow embryonic germ cells in tissue culture under appropriate conditions. Both EGCs and ESCs are pluripotent. For purpose of the present invention, the term “ESCs” is used broadly sometimes to encompass EGCs.

In some embodiments, iPSCs are derived by transfection of certain stem cell-associated genes into non-pluripotent cells, such as adult fibroblasts. Transfection is typically achieved through viral vectors, such as retroviruses. Transfected genes include the master transcriptional regulators Oct-3/4 (Pouf51) and Sox2, although it is suggested that other genes enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include but are not limited to first generation iPSCs, second generation iPSCs in mice, and human induced pluripotent stem cells. In some embodiments, a retroviral system is used to transform human fibroblasts into pluripotent stem cells using four pivotal genes: Oct3/4, Sox2, Klf4, and c-Myc. In alternative embodiments, a lentiviral system is used to transform somatic cells with OCT4, SOX2, NANOG, and LIN28. Genes whose expression are induced in iPSCs include but are not limited to Oct-3/4 (e.g., Pou5fl); certain members of the Sox gene family (e.g., Sox1, Sox2, Sox3, and Sox15); certain members of the Klf family (e.g., Klf1, Klf2, Klf4, and Klf5), certain members of the Myc family (e.g., C-myc, L-myc, and N-myc), Nanog, and LIN28.

More details on induced pluripotent stem cells can be found in, for example, Kaji et al., 2009, Nature 458:771-775; Woltjen et al., 2009, Nature 458:766-770; Okita et al., 2008, Science 322(5903):949-953; Stadtfeld et al., 2008, Science 322(5903):945-949; and Zhou et al., 2009, Cell Stem Cell 4(5):381-384.

In some embodiments, examples of iPS cell lines include but not limited to iPS-DF19-9; iPS-DF19-9; iPS-DF4-3; iPS-DF6-9; iPS(Foreskin); iPS(IMR90); and iPS(IMR90).

The present invention provides compositions comprising human intestinal tissue (derived from human pluripotent stem cells) comprising epithelial cells, stromal-fibroblast cells, neuronal cells, endothelial cells, organized smooth muscle, and ICCs similar to native human intestine tissue. In some embodiments, the smooth muscle expresses smooth muscle 22 (SM22) protein and/or transgelin (TAGLN) RNA. In some embodiments, the neuronal cells express TUBB3 and/or MAP2 at the protein and/or RNA level. In some embodiments, the endothelial cells express PECAM and/or CDH5 at the protein and/or RNA level. In some embodiments, the epithelial cells express CDX2 at the protein and/or RNA level. In some embodiments, the human intestinal tissue (derived from human pluripotent stem cells) is genetically engineered. In some embodiments, the human intestinal tissue comprises human intestinal organoid tissue. In some embodiments, the human intestinal tissue is human intestinal organoid tissue. In some embodiments, the human intestinal organoid tissue (derived from human pluripotent stem cells) is genetically engineered.

The present invention provides compositions comprising human intestinal organoid tissue (derived from human pluripotent stem cells) comprising epithelial cells, stromal-fibroblast cells, neuronal cells, endothelial cells, organized smooth muscle, and ICCs similar to native human intestine tissue. In some embodiments, the smooth muscle expresses smooth muscle 22 (SM22) protein and/or transgelin (TAGLN) RNA. In some embodiments, the neuronal cells express TUBB3 and/or MAP2 at the protein and/or RNA level. In some embodiments, the endothelial cells express PECAM and/or CDH5 at the protein and/or RNA level. In some embodiments, the epithelial cells express CDX2 at the protein and/or RNA level. In some embodiments, the human intestinal organoid tissue (derived from human pluripotent stem cells) is genetically engineered.

In certain embodiments, methods are provided comprising culturing hindgut spheroid tissue in vitro, wherein the culturing results in differentiation of the hindgut spheroid tissue into tissue comprising human intestinal tissue (e.g., human intestinal organoid tissue) comprising epithelial cells expressing CDX2, stromal-fibroblast cells, neuronal cells, endothelial cells, organized smooth muscle, and ICCs.

In certain embodiments, methods are provided comprising culturing hindgut spheroid tissue in vitro, wherein the culturing results in differentiation of the hindgut spheroid tissue into tissue comprising human intestinal tissue (e.g., human intestinal organoid tissue) comprising epithelial cells, stromal-fibroblast cells, neuronal cells, endothelial cells, organized smooth muscle, and ICCs.

In some embodiments, the human intestinal tissue (e.g., human intestinal organoid tissue) comprising epithelial cells, stromal-fibroblast cells, neuronal cells, endothelial cells, organized smooth muscle, and ICCs is similar to native human intestine tissue.

In some embodiments, the culturing of the hindgut spheroid tissue in vitro comprises culturing the hindgut spheroid tissue with media comprising EREG. In some embodiments, the concentration of EREG in the medium is between about 10 ng/mL and about 100 ng/mL (e.g., 10 ng/mL, 25 ng/mL, 50 ng/mL, or 100 ng/mL). In some embodiments, the media comprising EREG further comprises a BMP signaling pathway inhibitor selected from, for example, Noggin, dorsomorphin, and LDN-193189. In some embodiments, the media comprising EREG further comprises a Wnt signaling pathway activator selected from, for example, R-Spondin1, R-Spondin-2, R-Spondin-3, and CHIR99021. In some embodiments, the media comprising EREG further comprises a BMP signaling pathway inhibitor (e.g., Noggin, dorsomorphin, LDN-193189) and/or a Wnt signaling pathway activator (e.g., R-Spondin1, R-Spondin-2, R-Spondin-3, CHIR99021). In some embodiments, the media comprising EREG further comprises one or both of Noggin and R-Spondin1, and does not contain other members of the EGF family (e.g., EGF, NRG1). In some embodiments, the culturing of the hindgut spheroid tissue in vitro comprises culturing the hindgut spheroid tissue with media comprising EREG and not containing EGF or NRG1.

In some embodiments, the human intestinal tissue (e.g., human intestinal organoid tissue) is expanded and maintained. In some embodiments, the human intestinal tissue (e.g., human intestinal organoid tissue) is expanded and maintained with media comprising EREG. In some embodiments, the human intestinal tissue (e.g., human intestinal organoid tissue) is expanded and maintained with media comprising EREG and not containing EGF or NRG1. In some embodiments, smooth muscle expresses smooth muscle 22 (SM22) protein and/or transgelin (TAGLN) RNA. In some embodiments, the neuronal cells express TUBB3 and/or MAP2 at the protein and/or RNA level. In some embodiments, the endothelial cells express PECAM and/or CDH5 at the protein and/or RNA level. In some embodiments, the epithelial cells express CDX2 at the protein and/or RNA level.

In some embodiments, the duration of differentiating the hindgut spheroid tissue into tissue comprising human intestinal organoid tissue is approximately three days (e.g., 10 hours, 11 hours, 12 hours, 13 hours, 14 hours, 15 hours, 16 hours, 17 hours, 20 hours, 22 hours, 23 hours, 24 hours, 25 hours, 26 hours, 27 hours, 28 hours, 30 hours, 33 hours, 34 hours, 35 hours, 36 hours, 37 hours, 38 hours, 39 hours, 40 hours, 42 hours, 44 hours, 45 hours, 46 hours, 47 hours, 48 hours, 49 hours, 50 hours, 51 hours, 52 hours, 57 hours, 58 hours, 59 hours, 60 hours, 61 hours, 62 hours, 63 hours, 64 hours, 65 hours, 66 hours, 75 hours, 100 hours, 125 hours, 150 hours, 175 hours, 200 hours, 210 hours, 225 hours, 238 hours, 239 hours, 240 hours, 241 hours, 242 hours, 243 hours, 250 hours) (e.g., 0.5 days, 1 day, 1.5 days, 2 days, 2.5 days, 2.75 days, 3 days, 3.1 days, 3.5 days, 4 days, 5 days, 5.5 days).

In some embodiments, the hindgut spheroid tissue is obtained through culturing endoderm/mesoderm cells with a WNT signaling family activator (e.g., WNT3A, CHIR99021) and FGF4 for approximately six days (e.g., 30 hours, 33 hours, 34 hours, 35 hours, 36 hours, 37 hours, 38 hours, 39 hours, 40 hours, 42 hours, 44 hours, 45 hours, 46 hours, 47 hours, 48 hours, 49 hours, 50 hours, 51 hours, 52 hours, 57 hours, 58 hours, 59 hours, 60 hours, 61 hours, 62 hours, 63 hours, 64 hours, 65 hours, 66 hours, 75 hours, 100 hours, 125 hours, 150 hours, 175 hours, 200 hours, 210 hours, 225 hours, 238 hours, 239 hours, 240 hours, 241 hours, 242 hours, 243 hours, 250 hours, 300 hours, 350 hours, 400 hours, 450 hours, 500 hours, 550 hours, 600 hours) (e.g., 3 days, 4 days, 5 days, 6 days, 7 days, 8 days, 9 days). In some embodiments, the culturing results in spontaneous hindgut spheroid tissue formation. In some embodiments, the culturing results in hindgut spheroid tissue formation through cell-suspension re-aggregation.

In some embodiments, the endoderm/mesoderm cells are obtained through culturing induced pluripotent stem cells with Activin A for a period of approximately three days (e.g., 10 hours, 11 hours, 12 hours, 13 hours, 14 hours, 15 hours, 16 hours, 17 hours, 20 hours, 22 hours, 23 hours, 24 hours, 25 hours, 26 hours, 27 hours, 28 hours, 30 hours, 33 hours, 34 hours, 35 hours, 36 hours, 37 hours, 38 hours, 39 hours, 40 hours, 42 hours, 44 hours, 45 hours, 46 hours, 47 hours, 48 hours, 49 hours, 50 hours, 51 hours, 52 hours, 57 hours, 58 hours, 59 hours, 60 hours, 61 hours, 62 hours, 63 hours, 64 hours, 65 hours, 66 hours, 75 hours, 100 hours, 125 hours, 150 hours, 175 hours, 200 hours, 210 hours, 225 hours, 238 hours, 239 hours, 240 hours, 241 hours, 242 hours, 243 hours, 250 hours) (e.g., 1 day, 2 days, 3 days, 4 days, 5 days).

In some embodiments, the induced pluripotent stem cells are human induced pluripotent stem cells.

In some embodiments, the culturing steps are conducted in vitro.

In certain embodiments, methods are provided comprising a) culturing induced pluripotent stem cells (iPSCs) in a first culture medial comprising Activin-A to produce endoderm/mesoderm cells; b) treating the endoderm/mesoderm cells with a WNT signaling family activator (e.g., WNT3A, CHIR99021) and FGF4 to produce hindgut spheroid tissue; and c) contacting the hindgut spheroid tissue with a media comprising EREG to produce human intestinal tissue (e.g., human intestinal organoid tissue) comprising epithelial cells, stromal-fibroblast cells, neuronal cells, endothelial cells, organized smooth muscle, and ICCs (e.g., human intestinal tissue (e.g., human intestinal organoid tissue) comprising epithelial cells, stromal cells (also known as fibroblast cells), neuronal cells, endothelial cells, organized smooth muscle, and ICCs similar to native human intestine tissue). In some embodiments, the media comprising EREG within step c further comprises one or both of a BMP signaling pathway inhibitor (e.g., Noggin, dorsomorphin, LDN-193189) and/or a Wnt signaling pathway activator (e.g., R-Spondin1, R-Spondin-2, R-Spondin-3, CHIR99021). In some embodiments, the human intestinal tissue (e.g., human intestinal organoid tissue) is expanded and maintained. In some embodiments, the human intestinal tissue (e.g., human intestinal organoid tissue) is expanded and maintained with media comprising EREG.

In certain embodiments, kits are provided comprising human intestinal tissue (e.g., human intestinal organoid tissue) (derived from human pluripotent stem cells) comprising epithelial cells, stromal cells (also known as fibroblast cells), neuronal cells, endothelial cells, organized smooth muscle, and ICCs obtained through the methods described herein.

Such methods are not limited to a particular manner of accomplishing the directed differentiation of iPSCs into endoderm/mesoderm cells. Indeed, any method for producing endoderm/mesoderm cells from iPSCs is applicable to the methods described herein. In some embodiments, pluripotent cells are derived from a morula. Stem cells used in these methods can include, but are not limited to, embryonic stem cells. Embryonic stem cells can be derived from the embryonic inner cell mass or from the embryonic gonadal ridges. Embryonic stem cells or germ cells can originate from a variety of animal species including, but not limited to, various mammalian species including humans. In some embodiments, human embryonic stem cells are used to produce endoderm/mesoderm cells. In some embodiments, human embryonic germ cells are used to produce endoderm/mesoderm cells. In some embodiments, iPSCs are used to produce endoderm/mesoderm cells.

In some embodiments, one or more growth factors are used in the differentiation process from pluripotent stem cells to endoderm/mesoderm cells. The one or more growth factors used in the differentiation process can include Activin A.

In some embodiments, the embryonic stem cells or germ cells and iPSCs are treated with the one or more growth factors (e.g., Activin A) for 6 or more hours; 12 or more hours; 18 or more hours; 24 or more hours; 36 or more hours; 48 or more hours; 60 or more hours; 72 or more hours; 84 or more hours; 96 or more hours; 120 or more hours; 150 or more hours; 180 or more hours; or 240 or more hours (e.g., 30 hours, 33 hours, 34 hours, 35 hours, 36 hours, 37 hours, 38 hours, 39 hours, 40 hours, 42 hours, 44 hours, 45 hours, 46 hours, 47 hours, 48 hours, 49 hours, 50 hours, 51 hours, 52 hours, 57 hours, 58 hours, 59 hours, 60 hours, 61 hours, 62 hours, 63 hours, 64 hours, 65 hours, 66 hours, 75 hours, 100 hours, 125 hours, 150 hours, 175 hours, 200 hours, 210 hours, 225 hours, 238 hours, 239 hours, 240 hours, 241 hours, 242 hours, 243 hours, 250 hours, 300 hours, 350 hours, 400 hours, 450 hours, 500 hours, 550 hours, 600 hours).

In some embodiments, the embryonic stem cells or germ cells and iPSCs are treated with the one or more growth factors (e.g., Activin A) at a concentration of 10 ng/ml or higher; 20 ng/ml or higher; 50 ng/ml or higher; 75 ng/ml or higher; 100 ng/ml or higher; 120 ng/ml or higher; 150 ng/ml or higher; 200 ng/ml or higher; 500 ng/ml or higher; 1,000 ng/ml or higher; 1,200 ng/ml or higher; 1,500 ng/ml or higher; 2,000 ng/ml or higher; 5,000 ng/ml or higher; 7,000 ng/ml or higher; 10,000 ng/ml or higher; or 15,000 ng/ml or higher. In some embodiments, concentration of the growth factor is maintained at a constant level throughout the treatment. In other embodiments, concentration of the growth factor is varied during the course of the treatment. In some embodiments, the growth factor is suspended in media that include fetal bovine serine (FBS) with varying HyClone concentrations. One of skill in the art would understand that the regimen described herein is applicable to any known growth factors, alone or in combination. When two or more growth factors are used, the concentration of each growth factor may be varied independently.

In some embodiments, the endoderm/mesoderm cells are obtained through culturing induced pluripotent stem cells with Activin A for a period of approximately three days (e.g., 10 hours, 11 hours, 12 hours, 13 hours, 14 hours, 15 hours, 16 hours, 17 hours, 20 hours, 22 hours, 23 hours, 24 hours, 25 hours, 26 hours, 27 hours, 28 hours, 30 hours, 33 hours, 34 hours, 35 hours, 36 hours, 37 hours, 38 hours, 39 hours, 40 hours, 42 hours, 44 hours, 45 hours, 46 hours, 47 hours, 48 hours, 49 hours, 50 hours, 51 hours, 52 hours, 57 hours, 58 hours, 59 hours, 60 hours, 61 hours, 62 hours, 63 hours, 64 hours, 65 hours, 66 hours, 75 hours, 100 hours, 125 hours, 150 hours, 175 hours, 200 hours, 210 hours, 225 hours, 238 hours, 239 hours, 240 hours, 241 hours, 242 hours, 243 hours, 250 hours) (e.g., 1 day, 2 days, 3 days, 4 days, 5 days).

Methods for enriching a cell population with endoderm/mesoderm cells are also contemplated. In some embodiments, endoderm/mesoderm cells can be isolated or substantially purified from a mixed cell population by contacting the cells with a reagent that binds to a molecule that is present on the surface of endoderm/mesoderm cells but which is not present on the surface of other cells in the mixed cell population, and then isolating the cells bound to the reagent.

Additional methods for obtaining or creating endoderm/mesoderm cells that can be used in the present invention include but are not limited to those described in U.S. Pat. Nos. 7,510,876; 7,326,572; Kubol et al., 2004, Development 131:1651-1662; D'Amour et al., 2005, Nature Biotechnology 23:1534-1541; and Ang et al., 1993, Development 119:1301-1315.

In some embodiments, populations of cells enriched in endoderm/mesoderm cells are used. In some embodiments, the endoderm/mesoderm cells are isolated or substantially purified.

In some embodiments, directed differentiation toward hindgut spheroid tissue, and human intestinal tissue (e.g., human intestinal organoid tissue) is achieved by selectively activating certain signaling pathways in the iPSCs and/or endoderm/mesoderm cells. In some embodiments, the activated signaling pathways are those active in intestinal development in a step-wise manner.

In some embodiments, directed differentiation of endoderm/mesoderm cells into human intestinal tissue (e.g., human intestinal organoid tissue) is accomplished first through directed differentiation of endoderm/mesoderm cells into hindgut spheroid tissue, then directed differentiation of the hindgut spheroid tissue into human intestinal tissue (e.g., human intestinal organoid tissue).

Such techniques are not limited to a particular manner of inducing formation of hindgut spheroid tissue from endoderm/mesoderm cells. In some embodiments, inducing formation of hindgut spheroid tissue from endoderm/mesoderm cells is accomplished through culturing the endoderm/mesoderm cells with a WNT signaling family activator (e.g., WNT3A, CHIR99021) and FGF4 for approximately six days (e.g., 30 hours, 33 hours, 34 hours, 35 hours, 36 hours, 37 hours, 38 hours, 39 hours, 40 hours, 42 hours, 44 hours, 45 hours, 46 hours, 47 hours, 48 hours, 49 hours, 50 hours, 51 hours, 52 hours, 57 hours, 58 hours, 59 hours, 60 hours, 61 hours, 62 hours, 63 hours, 64 hours, 65 hours, 66 hours, 75 hours, 100 hours, 125 hours, 150 hours, 175 hours, 200 hours, 210 hours, 225 hours, 238 hours, 239 hours, 240 hours, 241 hours, 242 hours, 243 hours, 250 hours, 300 hours, 350 hours, 400 hours, 450 hours, 500 hours, 550 hours, 600 hours) (e.g., 3 days, 4 days, 5 days, 6 days, 7 days, 8 days, 9 days).

Such techniques are not limited to a particular manner of inducing formation of human intestinal tissue (e.g., human intestinal organoid tissue). In some embodiments, inducing formation of human intestinal tissue (e.g., human intestinal organoid tissue) from the hindgut spheroid tissue occurs through culturing the hindgut spheroid tissue with media comprising EREG. In some embodiments, inducing formation of human intestinal organoid tissue from the hindgut spheroid tissue occurs through culturing the hindgut spheroid tissue with media comprising EREG and one or both of Noggin and R-Spondin1. In some embodiments, culturing of the hindgut spheroid tissue in vitro occurs for approximately three days (e.g., 10 hours, 11 hours, 12 hours, 13 hours, 14 hours, 15 hours, 16 hours, 17 hours, 20 hours, 22 hours, 23 hours, 24 hours, 25 hours, 26 hours, 27 hours, 28 hours, 30 hours, 33 hours, 34 hours, 35 hours, 36 hours, 37 hours, 38 hours, 39 hours, 40 hours, 42 hours, 44 hours, 45 hours, 46 hours, 47 hours, 48 hours, 49 hours, 50 hours, 51 hours, 52 hours, 57 hours, 58 hours, 59 hours, 60 hours, 61 hours, 62 hours, 63 hours, 64 hours, 65 hours, 66 hours, 75 hours, 100 hours, 125 hours, 150 hours, 175 hours, 200 hours, 210 hours, 225 hours, 238 hours, 239 hours, 240 hours, 241 hours, 242 hours, 243 hours, 250 hours) (e.g., 0.5 days, 1 day, 1.5 days, 2 days, 3 days, 4 days, 5 days).

In some embodiments, the obtained human intestinal tissue (e.g., human intestinal organoid tissue) is maintained and expanded. In some embodiments, the obtained human intestinal tissue (e.g., human intestinal organoid tissue) is maintained and expanded through culturing the human intestinal organoid tissue with EREG for an extended period of time (e.g., 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 30 days, 40 days, 50 days, 75 days, 100 days).

In some embodiments, human intestinal tissue (e.g., human intestinal organoid tissue) produced in vitro from the described methods can be used to screen drugs for intestinal tissue uptake and mechanisms of transport. For example, this can be done in a high throughput manner to screen for the most readily absorbed drugs, and can augment Phase 1 clinical trials that are done to study drug intestinal tissue uptake and intestinal tissue toxicity. This includes pericellular and intracellular transport mechanisms of small molecules, peptides, metabolites, salts.

In some embodiments, human intestinal tissue (e.g., human intestinal organoid tissue) produced in vitro from the described methods can be used to identify the molecular basis of normal human intestinal development.

In some embodiments, human intestinal tissue (e.g., human intestinal organoid tissue) produced in vitro from the described methods can be used to identify the molecular basis of congenital defects affecting human intestinal development.

In some embodiments, human intestinal tissue (e.g., human intestinal organoid tissue) produced in vitro from the described methods can be used to correct intestinal related congenital defects caused by genetic mutations. In particular, mutation affecting human intestinal development can be corrected using iPSC technology and genetically normal vascularized human intestinal organoid tissue produced in vitro from the described methods.

In some embodiments, human intestinal organoid tissue produced in vitro from the described methods can be used to generate replacement tissue.

In some embodiments, human intestinal tissue (e.g., human intestinal organoid tissue) produced in vitro from the described methods can be used to generate replacement intestinal tissue for intestine related disorders.

In some embodiments, a diagnostic kit or package is developed to include human intestinal tissue (e.g., human intestinal organoid tissue) produced in vitro from the described methods and based on one or more of the aforementioned utilities.

The invention provides a composition comprising a culture medium according to the invention and stem cells. The invention also provides a composition comprising a culture medium according to the invention and organoids. Furthermore, the invention provides a composition comprising a culture medium according to the invention. Furthermore, the invention provides a composition comprising a culture medium according to the invention and an extracellular matrix.

The invention also provides a composition comprising a culture medium of the invention, an extracellular matrix and human pluripotent stem cells. The invention also provides a composition comprising a culture medium of the invention, an extracellular matrix and human intestinal tissue (e.g., human intestinal organoid tissue) of the invention. In some embodiments, the human pluripotent stem cells are embryonic stem cells. In some embodiments, the human pluripotent stem cells are induced pluripotent stem cells.

The invention also provides a hermetically-sealed vessel containing a culture medium of the invention. Hermetically-sealed vessels may be preferred for transport or storage of the culture media or culture media supplements disclosed herein, to prevent contamination. The vessel may be any suitable vessel, such as a flask, a plate, a bottle, ajar, a vial or a bag.

The invention provides the use of human intestinal tissue (e.g., human intestinal organoid tissue) of the invention or cells derived thereof in drug screening, (drug) target validation, (drug) target discovery, toxicology and toxicology screens, personalized medicine, regenerative medicine and/or as ex vivo cell/organ models, such as disease models.

Cells and vascularized human intestinal tissue (e.g., human intestinal organoid tissue) cultured according to the media and methods of the invention are thought to faithfully represent the in vivo situation. This is true both for expanded populations of cells and organoids grown from normal tissue and for expanded populations of cells and organoids grown from diseased tissue. Therefore, as well as providing normal ex vivo cell/organ models, the organoids of the invention can be used as ex vivo disease models.

Organoids of the invention (e.g., human intestinal organoid tissue produced with the methods described herein) can also be used for culturing of a pathogen and thus can be used as ex vivo infection models. Examples of pathogens that may be cultured using an organoid of the invention include viruses, bacteria, prions or fungi that cause disease in its animal host. Thus an organoid of the invention can be used as a disease model that represents an infected state. In some embodiments of the invention, the organoids can be used in vaccine development and/or production.

Diseases that can be studied by the organoids of the invention (e.g., human intestinal organoid tissue produced with the methods described herein) thus include genetic diseases, metabolic diseases, pathogenic diseases, inflammatory diseases etc of the intestine and/or related to intestinal development.

The organoids of the invention (e.g., human intestinal organoid tissue produced with the methods described herein) can be frozen and thawed and put into culture without losing their genetic integrity or phenotypic characteristics and without loss of proliferative capacity. Thus, the organoids can be easily stored and transported. Thus, in some embodiments, the invention provides a frozen organoid.

For these reason the organoids or expanded populations of cells of the invention can be a tool for drug screening, target validation, target discovery, toxicology and toxicology screens and personalized medicine.

Accordingly, in a further aspect, the invention provides the use of an organoid or cell derived from said organoid according to the invention in a drug discovery screen, toxicity assay or in medicine, such as regenerative medicine. For example, the human intestinal organoid tissue may be used in a drug discovery screen, toxicity assay or in medicine, such as regenerative medicine.

For preferably high-throughput purposes, said organoids of the invention (e.g., human intestinal organoid tissue produced with the methods described herein) are cultured in multiwell plates such as, for example, 96 well plates or 384 well plates. Libraries of molecules are used to identify a molecule that affects said organoids. Preferred libraries comprise antibody fragment libraries, peptide phage display libraries, peptide libraries, lipid libraries, synthetic compound libraries or natural compound libraries. Furthermore, genetic libraries can be used that induce or repress the expression of one of more genes in the progeny of the stem cells. These genetic libraries comprise cDNA libraries, antisense libraries, and siRNA or other non-coding RNA libraries. The cells are preferably exposed to multiple concentrations of a test agent for a certain period of time. At the end of the exposure period, the cultures are evaluated. The term “affecting” is used to cover any change in a cell, including, but not limited to, a reduction in, or loss of, proliferation, a morphological change, and cell death.

In some embodiments, the organoids of the invention (e.g., human intestinal organoid tissue produced with the methods described herein) can be used to test libraries of chemicals, antibodies, natural product (plant extracts), etc for suitability for use as drugs, cosmetics and/or preventative medicines.

The invention provides the use of human intestinal organoid tissue in regenerative medicine and/or transplantation. The invention also provides methods of treatment wherein the method comprises transplanting an organoid into an animal or human.

Human intestinal organoid tissue are useful in regenerative medicine, for example in treatment of post-radiation and/or post-surgery repair of the intestinal epithelium, in the repair of the intestinal epithelium in patients suffering from inflammatory bowel disease such as Crohn's disease and ulcerative colitis, and in the repair of the intestinal epithelium in patients suffering from short bowel syndrome. Further use is present in the repair of the intestinal epithelium in patients with hereditary diseases of the small intestine/colon.

### EXAMPLES

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention.

### Example 1

This example describes generation of human intestinal organoids containing neurons, endothelial cells, organized smooth muscle, and interstitial cells of Cajal (ICCs).

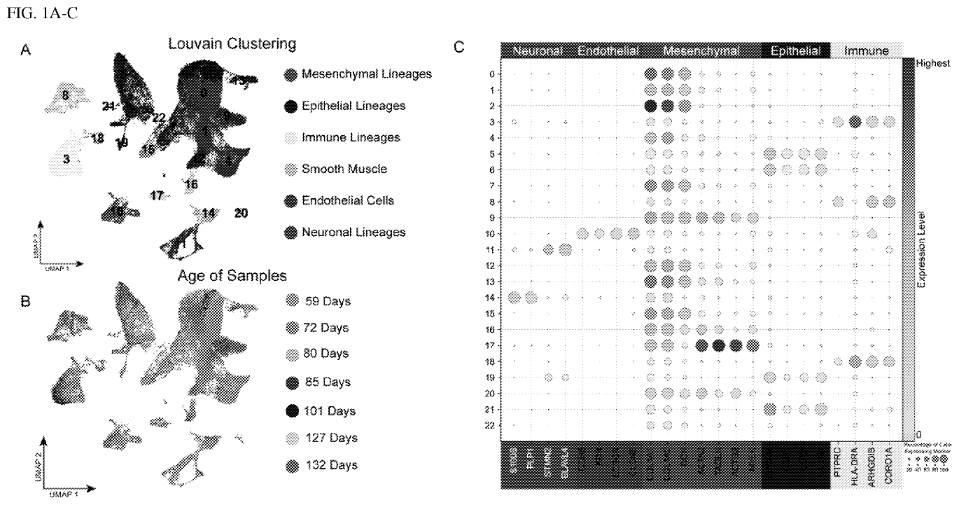

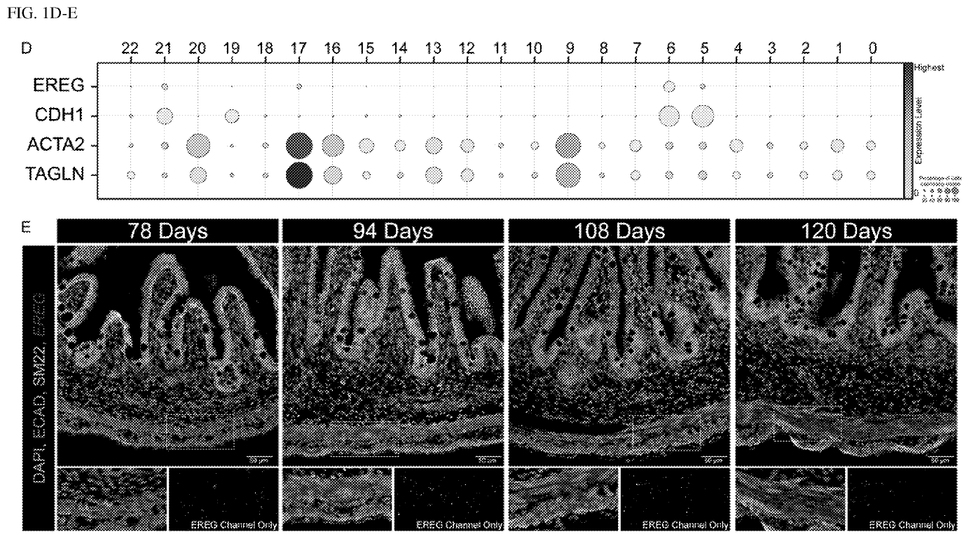

The directed differentiation method to generate HIOs from human iPSCs is robust and has been widely used for over a decade^{1,16}. Broadly, this differentiation approach relies on the induction of a mixed endoderm-mesoderm culture followed by further differentiation into intestinal lineages. During intestinal lineage differentiation, cells growing in 2-dimensional (2D) culture self-organize and form 3-dimensional (3D) spheroids that possess cells derived from endoderm and mesoderm. Spheroids are typically placed in media containing EGF, NOGGIN, and RSPONDIN-1 (ENR Media) for 3 days, which patterns a proximal, small-intestinal identity^{11,17}, and can then be cultured in media containing only EGF. These organoids have been extensively characterized^{1,6,11,18}; single cell RNA-sequencing (scRNA-seq) has revealed that small populations of neuron-like and endothelial-like cells are present in early cultures, but these populations are transient^{11,19} Smooth muscle cells can be found in these organoids, but they are rare, sparsely distributed and unorganized within the HIOs in vitro²⁰. It was recently reported that EPIREGULIN (EREG) is a stem cell niche factor in vivo during human intestinal development, and can replace EGF in tissue-derived intestinal enteroid culture, leading to improved spatial organization and enhanced differentiation of all epithelial cell types¹⁵. A more comprehensive analysis of the previously published scRNA-seq data followed by validation with fluorescence in situ hybridization (FISH) shows that EREG is also expressed in the outer longitudinal and circular smooth muscle of the developing human intestine at all time points examined (FIG. 1a-e). Based on these new observations and prior findings that EGF is expressed in the differentiated villus epithelial cells, it was hypothesized that EREG may play a role in mesenchymal patterning and differentiation during intestinal development.

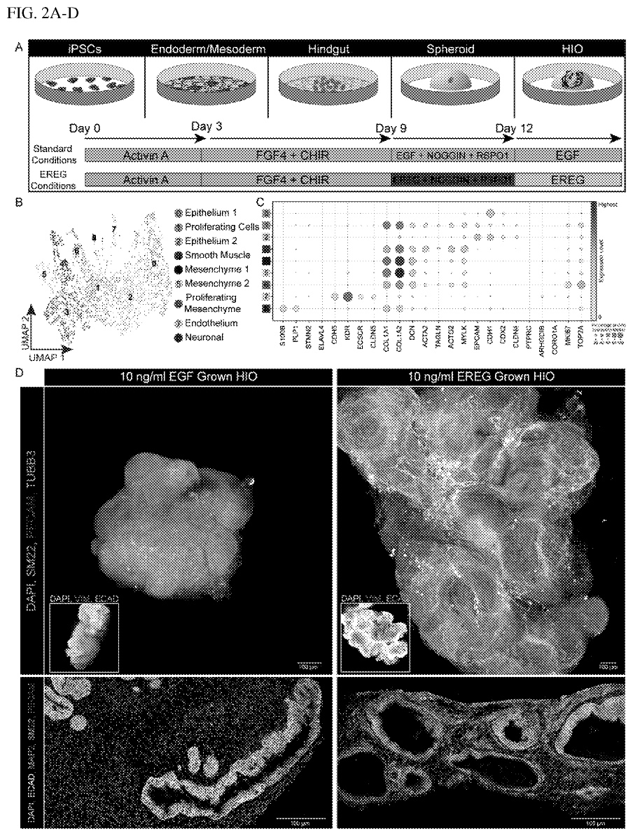

To test if EREG plays a role during HIO differentiation, the previously described differentiation paradigm (FIG. 2a) was followed up to the spheroid stage^{1,16}. Once spheroids are collected and embedded in Matrigel, they are patterned for 3 days with ENR media, and subsequently cultured in EGF-only media, at a standard concentration of 100 ng/mL. To determine if EREG can replace EGF in culture and retain differentiation potential, spheroids were cultured in three concentrations of EREG (1 ng/mL, 10 ng/mL, 100 ng/mL) and compared this to EGF (1 ng/mL, 10 ng/mL, 100 ng/mL), with a constant concentration of NOGGIN and RSPO1 for the first three days, before switching to EREG-only or EGF-only media (FIG. 2a).

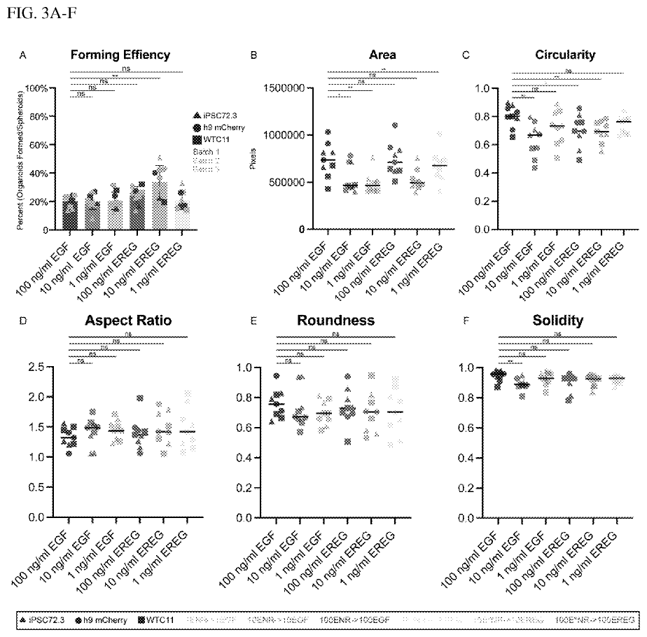

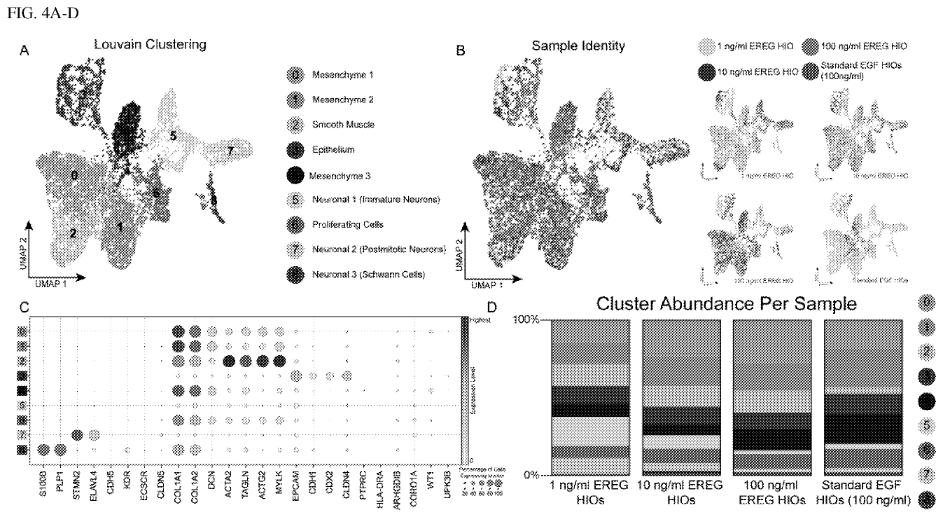

It was determined the HIO forming efficiency of each concentration by counting the number of spheroids plated in a single well at day 0, and again counting the number of HIOs present after 10 days in culture (formula: # of day 10 HIOs/# of day 0 spheroids=forming efficiency). Experiments were repeated for 3 separate experiments (batches), in 3 different iPSC lines with 3 technical replicates. Minimal difference in HIO forming efficiency was observed when comparing ligand or concentration, with growth in 10 ng/mL EREG having the only statistically significant increase in forming efficiency across conditions compared to the EGF control cultures (FIG. 3a). Morphologic features between the culture conditions were compared, including area, circularity, roundness, solidity, and aspect ratio. In general, any consistent patterns or major differences in these measurements between doses or media were observed (FIG. 3b-f), suggesting all EREG concentrations form HIOs similar in shape and size as the EGF controls.

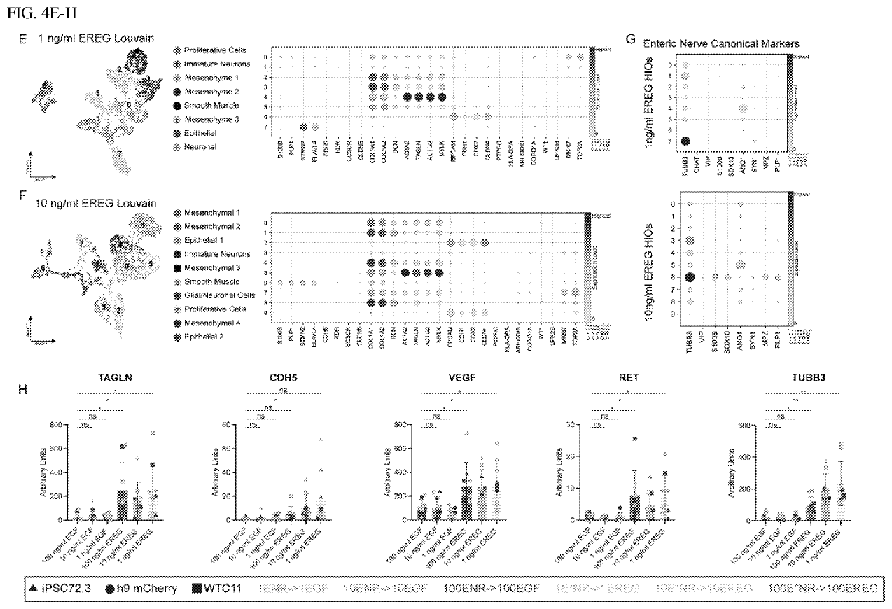

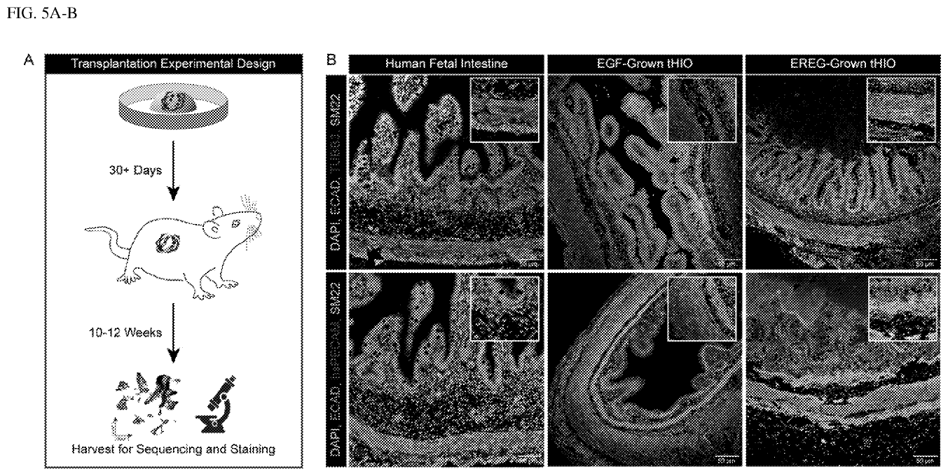

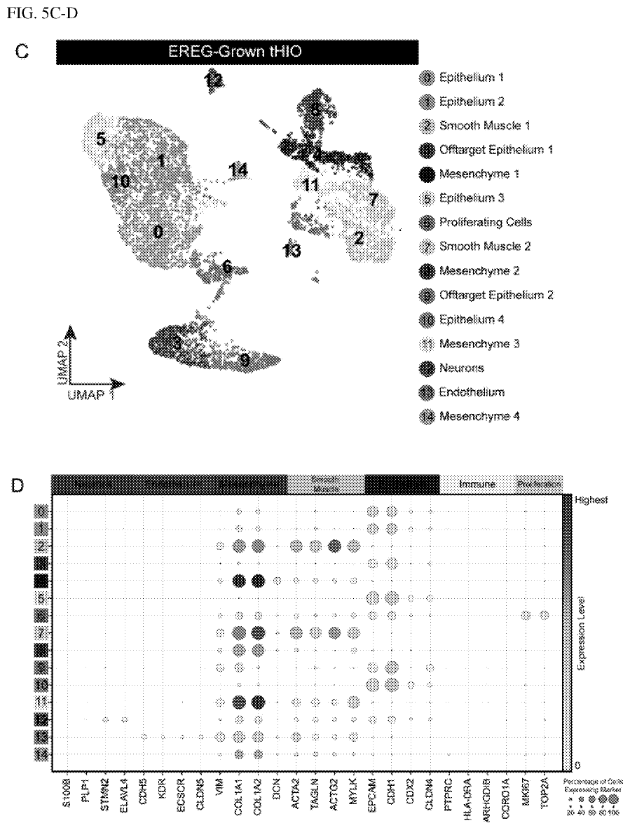

To characterize HIOs, all three concentrations of EREG-grown HIOs were subjected to scRNA-seq (FIG. 4a-b) and a cluster was found with enriched with smooth muscle genes (cluster 2: ACTA2, TAGLN, ACTG2, MYLK) and 3 clusters were found with neuroglial identities (clusters 5, 7, and 8: S100B, PLP1, STMN2, ELAVL4); which have been found in rare, transient populations in EGF-cultured HIOs previously^{11,12,18} (FIG. 4c). When cluster abundance were plotted per sample, it was found that most of the cells contributing to smooth muscle and neural clusters were from the EREG samples, with the 1 ng/mL and 10 ng/mL samples having the highest contributions to these lineages (FIG. 4d). The 10 ng/mL EREG condition possessed both glial and neuronal populations (FIG. 4e-f) and featured the most heterogeneous enteric neuron populations (FIG. 4g). Based on the high HIO forming efficiency (FIG. 5a) and the robust neural cell types found within the 10 ng/mL EREG condition, this condition was chosen for further characterization.

It was confirmed that enhanced differentiation of neurons and smooth muscle in EREG culture compared to EGF culture, across three different experiments, and in three different iPSC lines using RT-qPCR (FIG. 4h). During this validation, endothelial gene expression signatures (CDH5, VEGF) in all 3 biological replicates examined by RT-qPCR (FIG. 4h) was observed. Interestingly, no endothelial cells were observed in the whole cell scRNA-seq data. However, endothelial cells are known to be sensitive to dissociation methods and are often lost during preparation of samples for whole cell sequencing²¹. Thus, nuclei was isolated from the 10 ng/mL EREG HIOs and single nuclear RNA sequencing (snRNA-seq) data generated, which supported the presence of endothelial cells (FIG. 2b-c). Notably, fewer neurons were present in snRNA-seq data, as previously reported^{22,23}. Experiments next validated the presence of neural, endothelial, and smooth muscle lineages with whole mount 3D immunostaining and in 2D sections of HIOs grown in EGF or EREG, and observed that EREG-grown, but not EGF-grown HIOs, possessed organized SM22⁺ (TAGLN⁺) smooth muscle, networks of TUBB3⁺ (3D) and MAP2⁺ (2D) neuron-like cells, and PECAM⁺ endothelial cells throughout the HIOs (FIG. 2d).

Experiments further demonstrated that ICCs were identified within the smooth-muscle compartment of EREG-grown organoids. The ICCs function as the gut's pacemaker, generating rhythmic electrical “slow waves” that set the timing for contractions. The enteric neurons act as the control center, responding to the presence of food by sending specific signals to contract (behind the food) and relax (in front of it). Finally, the smooth muscle cells are the engine, performing the actual contraction only when they receive both the excitatory command from the neurons and the permissive pacemaker signal from the ICCs. Dysfunction of this triad can lead to severe motility disorders.

### Example 2

This example demonstrates that transplantation of EREG-HIOs leads to increased maturation and cellular organization.

Murine kidney capsule engraftment is well established as a system to further mature in vitro grown HIOs^{2,5,13,20,24,25}. In short, HIOs grown in vitro for 28 days or more are surgically placed under the kidney capsule of immunocompromised NSG mice, where they engraft and grow for an additional 10-12 weeks. These organoids greatly expand in size, mature to become spatially organized similar to the native human intestine, have increased cell type diversity, and can be harvested for snRNA-sequencing and staining¹⁹ (FIG. 5a). To determine if EREG-grown HIOs could be further matured and become functional, experiments were conducted that transplanted 10 ng/mL EREG- or 10 ng/mL EGF-grown control HIOs beneath the kidney capsule and allowed them to grow for 12 weeks. Immunofluorescent (IF) staining for epithelial (ECAD) and smooth muscle (TAGLN) markers revealed that both EGF-transplanted HIOs (tHIOs) and EREG-tHIOs were spatially organized as previously reported with the development of crypt-villus units and organized smooth muscle (FIG. 5b). Very few neurons or endothelial cells in control EGF-tHIOs (FIG. 5b, middle panels) were observed, while EREG-tHIOs possessed abundant TUBB3⁺ neurons and PECAM⁺ endothelial cells spatially organized similar to the native human intestine (FIG. 5b).

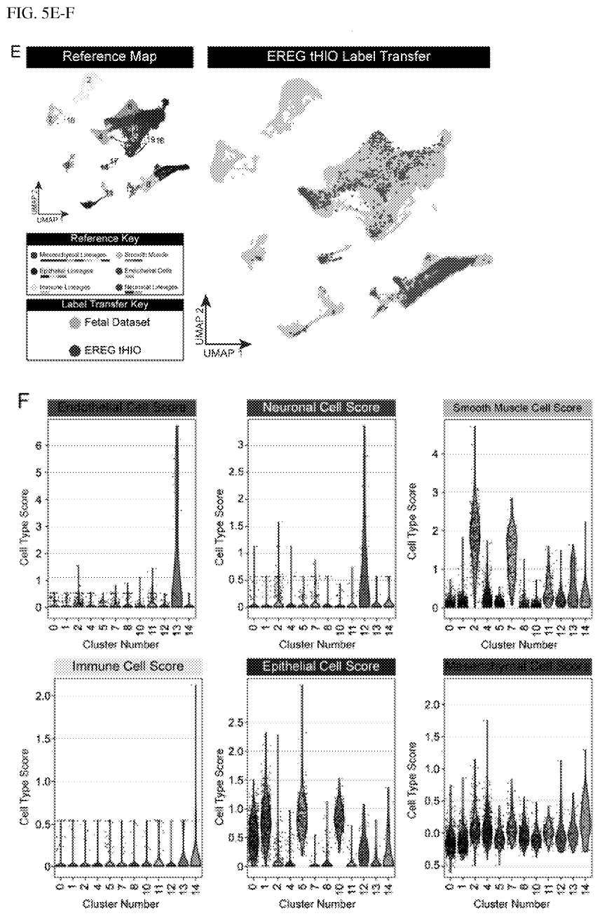

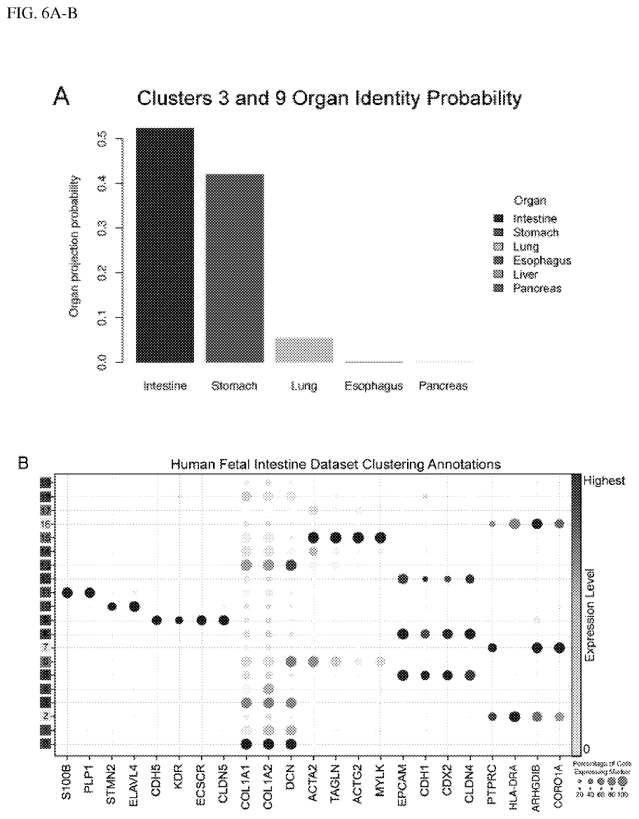

SnRNA-sequencing of EREG-tHIOs confirmed the presence of epithelial cells, mesenchymal cells/fibroblasts, smooth muscle, neural cells, and endothelial cells (FIG. 5c-d). Two undefined epithelial clusters (clusters 3 and 9) were found and appeared to be mixed gastric and intestinal epithelium based on mapping to a human fetal endoderm atlas¹⁹ (FIG. 6a). Small portions of undefined or non-intestinal cells have previously been reported in HIOs¹⁹, and these cells were excluded from further analysis. To confirm cluster annotations based on marker gene expression (FIG. 5d), a label transfer of EREG-tHIO data was carried out onto a previously published fetal intestine reference dataset²⁶ (FIG. 5e and Fig. Ta), and confirmed the accuracy of annotations based on marker genes. tHIO datasets robustly label transferred onto their counterparts in the human fetal datasets using Seurat's integrated label transfer function²⁷ (FIG. 5e, FIG. 6b). To further benchmark tHIO cell type identity against the reference dataset, a cohort of highly enriched genes for each major cell type in the reference was used (endothelial, neural, smooth muscle, immune, epithelial, and mesenchymal), that allowed generation of a “score” for each cell in the tHIO sample based on the enrichment of these genes. Cluster 6 contained both proliferating mesenchymal and epithelial cells, it was excluded from this analysis for clarity. It was found that the annotated tHIO clusters scored highly for their counterparts in the reference set, further confirming the annotations and the label transfer findings (FIG. 5f, FIG. 6b, Table 1). Collectively, this data indicates the cell types within tHIOs, including endothelial cells and neurons, share transcriptional states with their analogous cell type within the human intestine.

TABLE 1
Detected Gene Expression in Respective Cells

| Smooth | | | | | |
|---|---|---|---|---|---|
| Muscle | Immune | Epithelium | Mesenchyme | Neural | Endothelium |
| WIF2 | PTPRC | SMIM22 | C1QTNF3 | PHOX2A | CDH5 |
| MYH11 | C1QC | PHGR1 | ADH1B | PRPH | ECSCR |
| ACTG2 | IGSF6 | C19orf33 | SHISA3 | STMN4 | TM4SF18 |
| GREM2 | C1QB | CLDN3 | COL1A1 | EEF1A2 | MYCT1 |
| DES | HLA-DQA2 | PRAP1 | OGN | NRSN1 | FLT1 |
| CNN1 | HLA-DQA1 | CDX2 | RPS7 | INA | NRN1 |
| TAGLN | CSF1R | SLC26A3 | H3F3B | TTC9B | ROBO4 |
| RGS5 | C1QA | EPCAM | MALAT1 | STMN2 | ARHGEF15 |
| ACTA2 | HLA-DRB1 | USH1C | ITGB1 | GNG3 | CLDN5 |
| MYLK | P2RY13 | CCL15 | PTMS | TLX2 | TIE1 |
| JPH2 | EB13 | SMLR1 | PBK | ELAVL3 | VWF |
| HSPB7 | MS4A6A | CHP2 | KIF20A | KIF5A | SOX7 |
| HAND1 | HLA-DRB5 | S100A14 | SPC25 | GAP43 | MMRN2 |
| BTC | CLEC10A | GOLT1A | CKAP2L | GAL | BCL6B |
| NRXN3 | MS4A4A | FABP2 | TOP2A | TAGLN3 | MADCAM1 |
| KCNMB1 | HLA-DPA1 | UGT2A3 | COX4I2 | ELAVL4 | ESAM |
| SBSPON | MPEG1 | ESPN | KRT25 | TUBB2B | KANK3 |
| MYL9 | HLA-DRA | CLRN3 | ACKR4 | GDAP1L1 | FAM167B |
| CPXM2 | FOLR2 | CDX1 | GALR2 | CHRNA3 | KDR |
| LMOD1 | HLA-DQB1 | GJB1 | KCNJ8 | DPP6 | SOX18 |

### Example 3

This example demonstrates that EREG-tHIOs exhibit peristaltic-like function.

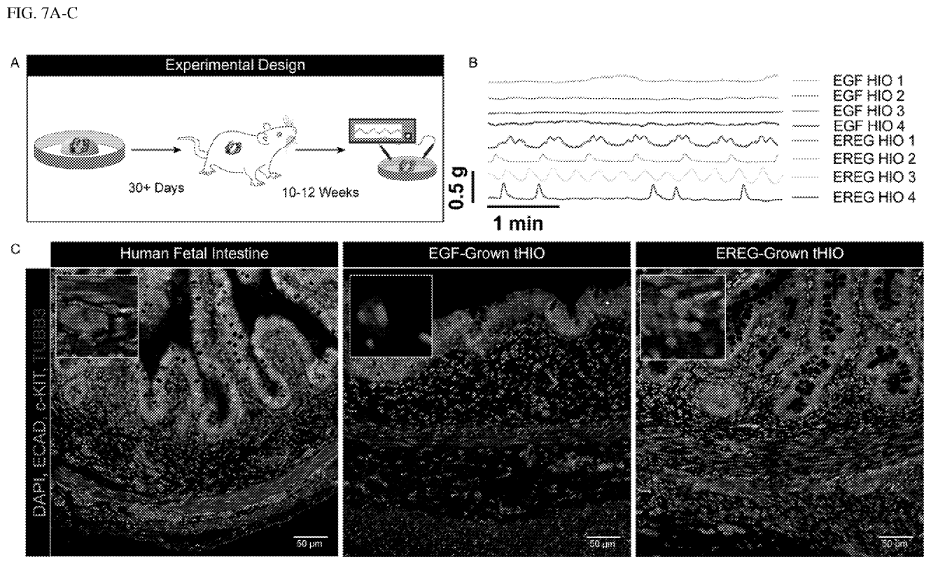

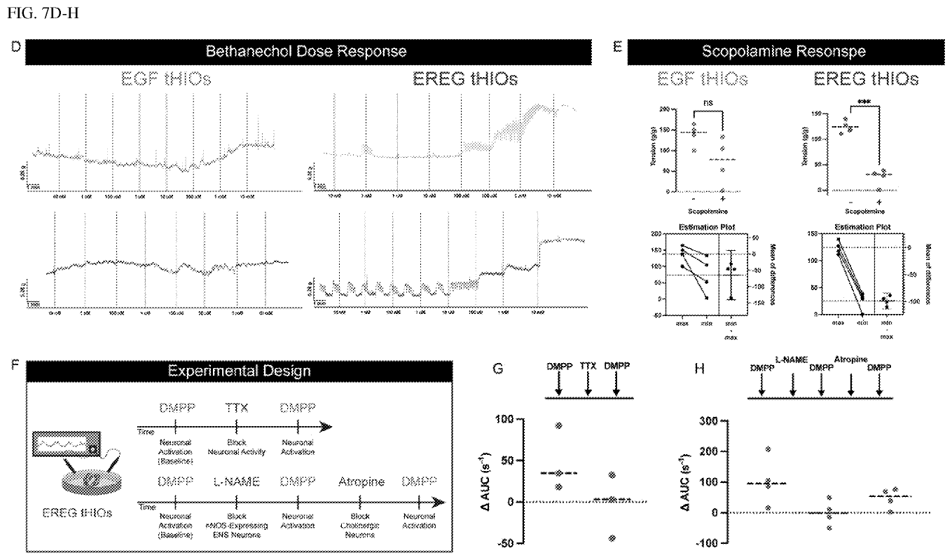

To test if the neurons and smooth muscle seen within EREG-tHIOs create a functional neuromuscular unit, control EGF-tHIOs and EREG-tHIOs were dissected into small muscle strips and explanted into Kreb's buffer in an organ bath chamber to measure muscle contractile force, as previously reported^{12,28}. Explanted muscle strips were allowed to equilibrate and monitored continuously for contractions (FIG. 7a). In the absence of any stimulation, spontaneous and rhythmic contractions were observed in EREG-tHIOs, a phenomenon that was not seen in control EGF-tHIOs (FIG. 7b). These phasic contractions indicate the presence of intramuscular interstitial cells of Cajal (ICCs) in EREG-grown tHIOs^{12,29}. The neuronal marker TUBB3 and the ICC marker c-KIT were each stained and it was found that EGF-tHIOs had rare TUBB3⁺ neurons and completely lacked c-KIT⁺ ICCs. On the other hand, EREG-tHIOs contained many c-KIT⁺ ICCs directly adjacent to TUBB3⁺neurons, closely resembling the staining pattern found in the neural plexus of the developing human intestine (FIG. 7c).

Next, the organoids were treated with bethanechol, a muscarinic receptor agonist that directly stimulates muscle contractions, and contractile force measured. No notable change in contractile force could be measured in control EGF-tHIOs (FIG. 7d—left, grey) while contractile force increased in a dose-dependent manner in response to bethanechol in EREG-tHIOs (FIG. 7d—right, pink). Experiments next treated the explanted tHIOs with scopolamine, a muscarinic antagonist that blocks smooth muscle contraction, and were able to trigger significant muscle relaxation (FIG. 3e—right, pink) in EREG-tHIOs but not in EGF-tHIOs (FIG. 7e—left, grey). With these data suggesting functional muscle and the presence of neurons in these organoids, the presence of a functional neuromuscular unit in EREG-tHIOs was hypothesized.

To examine if the neuronal populations functionally regulate smooth muscle contractions, neurons were excited by using the selective a3-nicotinic receptor agonist dimethylphenylpiperazinium (DMPP) to stimulate neurotransmitter release and activate neurons in EREG-grown tHIOs (FIG. 7f). Following DMPP treatment, explants were treated with tetrodotoxin (TTX) to block action potentials which successfully blocked the neurons' ability to be depolarized again following another dose of DMPP, thus supporting ENS-dependent contractile activity within the tissue (FIG. 7g). Finally, the function of nNOS-expressing neurons was assessed by inhibiting them with NG-nitro-L-arginine methyl ester (L-NAME), and cholinergic neurons by blocking them with a dose of atropine. Contractile activity following a baseline stimulation or stimulation after exposure to either inhibitor was next measured (FIG. 3h). Contractile activity was measured as the change in the area under the curve (ΔAUC) immediately before and after each stimulation. After each inhibitor was added, a significant decrease in muscle relaxation was observed compared to the uninhibited contractions (FIG. 7h), indicating nNOS-expressing neurons and cholinergic neurons elicit smooth contractions in EREG-tHIO explants. These data together demonstrate EREG-tHIOs not only possess glial, neuronal, and smooth muscle populations but that these populations are functional and collectively drive peristaltic smooth muscle-like contractions.

### Example 4

This example demonstrates that EREG-HIOs possess endothelial cells that organize into functional vasculature.

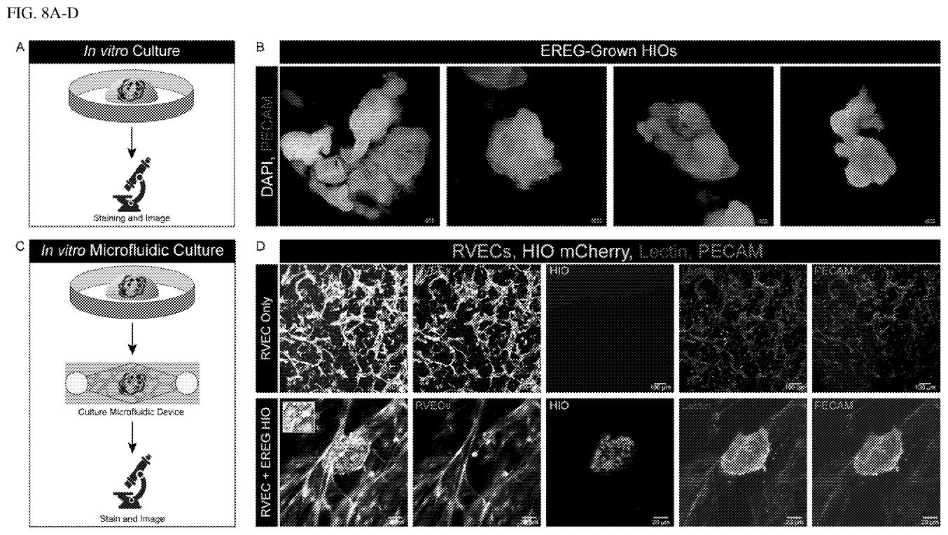

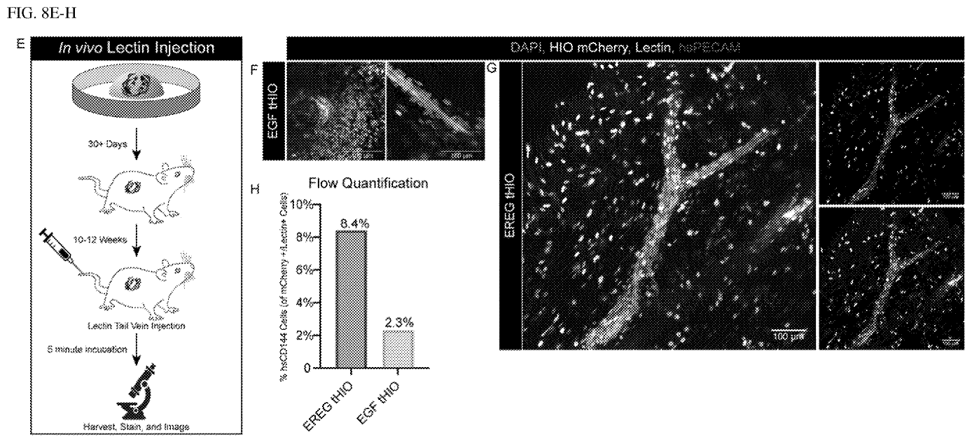

Tissue-specific endothelial cells are a critical cell type in all organs as they not only support the metabolic demands of a particular organ, but also supply paracrine angiocrine factors that orchestrate organ development, repair, and regeneration. Corroborating the single nucleus data (FIG. 5b-c), whole mount IF staining was used to interrogate many individual EREG-HIOs and robust endothelial cells networks throughout the organoids was consistently observed (FIG. 8a-b). As with the presence of endogenous neurons and smooth muscle structures, understanding if these endothelial cells could form functional vessels was next examined.

To test their tubulogenic capabilities in vitro, experiments were conducted that leveraged human ‘reset-vascular endothelial cells’ (RVECs) which have been shown to self-assemble into stable, multilayered and branching perfusable, vascular networks within scalable microfluidic chambers, which are capable of transporting human blood, and vascularizing colonic enteroids³⁰ (FIG. 8c). R-VECs are engineered by transient introduction of the pioneer transcription factor ETV2 into human endothelial cells, conferring them with the capacity to respond to biophysical and biochemical signals emanating from the microenvironment, such as intestinal epithelial cells. It was hypothesized that RVECs would adapt and anastomose to the endogenous endothelial cells within the EREG-HIO, enabling flow through the HIO. To this end, iPSCs labeled with a lentivirus expressing nuclear mCherry were used to derive mCherry⁺ EREG-HIOs. After 14 days in culture, mCherry⁺ EREG-HIOs were mixed with GFP-labeled RVECs in a fibrin matrix and seeded into a microfluidic device. After 48 hours, a tomato lectin dye and PECAM antibody were flowed through the system to label any vascular networks that had formed³¹. GFP⁺ RVECs anastomosing to mCherry⁺/PECAM⁺/Lectin⁺ networks were observed within the EREG-HIOs indicating that endogenous endothelial cells were not only able to connect to the RVEC network but that they formed perfusable vessel-like structures that enabled flow of media and lectin (FIG. 8d).

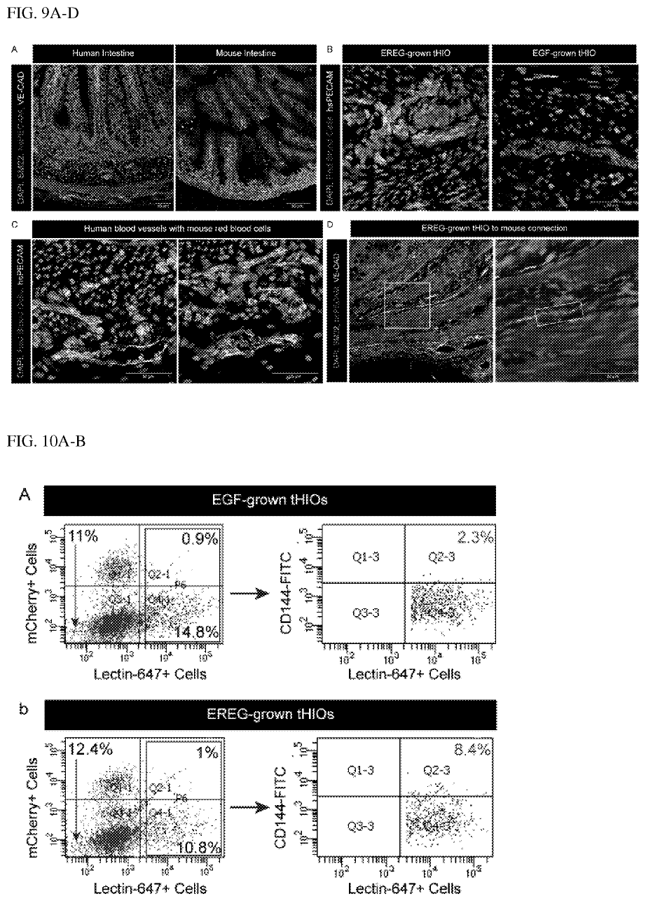

To interrogate the function of endothelial cells within EREG-HIOs in vivo, EREG-HIOs were transplanted under the kidney capsule of a mouse and allowed to grow for 10 weeks. These organoids were harvested and co-stained with a human-specific PECAM (hsPECAM) antibody and a pan-VE-CAD antibody that cross reacts with both human and mouse to confirm species specificity of observed endothelial cells (FIG. 9a). Within EGF-tHIOs, only rare human endothelial cells labeled with the hsPECAM antibody were noticed.

In contrast, EREG-tHIOs contained abundant hsPECAM labeling (FIG. 9b). It was also observed that murine red blood cells autofluoresce in the 488-channel, so this autofluorescence was leveraged to show that hsPECAM⁺ vascular structures within EREG-tHIOs were filled with mouse red blood cells (FIG. 9c), strongly suggesting that EREG-tHIO endothelial cells anastomose with the host's circulatory system. Based on hsPECAM/VE-CAD co-labeling, multiple points where a mouse blood vessel were observed (VE-CAD⁺/hsPECAM^{neg}), which appeared to connect to a human blood vessel (PECAM⁺/VE-CAD⁺) further suggesting that EREG-tHIO endothelial cells anastomose with the mouse circulatory system to form functional vessels (FIG. 9d).

To directly test a functional connection between the murine and EREG-tHIO vasculature, organoids were transplanted into a murine host and allowed them to mature for 10 weeks (FIG. 8e). The host was then injected with tomato-lectin via tail vein injection³⁰ and allowed the lectin to circulate for 5 minutes. Organoids were then harvested and whole mount IF stained with the hsPECAM antibody to delineate between mouse and human endothelial cells (FIG. 8e). Within EGF-tHIOs, only small rare Lectin⁺ staining of vascular-like structures was found, which were largely hsPECAM-negative, suggesting that most vessels in these tHIOs were host-derived (FIG. 8f). On the other hand, in EREG-tHIOs, large mCherry⁺/hsPECAM⁺/Lectin⁺ vessels were readily observed (FIG. 8g). Lectin staining in mCherry⁺/hsPECAM⁺ structures suggest these human vessels are connected to the host's circulatory system. To quantify the proportion of human-specific endothelial cells labeled by lectin, these transplanted organoids were dissociated after lectin injection and flow cytometry used to determine the proportion of mCherry⁺ human cells that were also positive for a VE-CAD flow antibody (CD144)⁺/Lectin⁺ in both EGF-tHIOs and EREG-tHIOs. It was found that EREG-tHIOs had a much larger proportion of human blood vessels labeled with lectin (8.4%) compared to EGF-tHIOs (2.3%) (FIG. 8h and FIG. 10a-b). These data taken together demonstrate EREG-grown HIO's endogenous endothelial population is able to connect to circulatory systems both in vitro and in vivo and have the ability to enable function perfusion to sustain their viability and function.

### Example 5

This example provides a discussion related to Examples 1-4.

By leveraging information from the developing human intestine, which allowed identification of the developmental niche factor EREG, the first hPSC-derived human intestinal organoid model was generated that can simultaneously pattern epithelial, mesenchymal/stromal, neural, endothelial and smooth muscle populations. By transplanting these organoids into a murine host, HIOs were expanded and matured further in order to assess function. EREG-tHIOs not only contain neurons and endothelial cell populations, but they also function in vivo. Functional experiments to interrogate smooth muscle contractions strongly support the development of a functional neuromuscular unit between neural populations and smooth muscle populations; similarly, several lines of evidence support that HIO-derived endothelial cells within the organoid connect with in vitro and in vivo circulatory systems, creating vasculature capable of flow.

Taken together, these results demonstrate the ability to create more physiologically relevant culture conditions to pattern complex and accurate organoid models of the human intestine. This system improves complexity and may enhance the utility of HIOs to understand human intestinal development, disease modeling, drug screening, and personalized medicine.

### Example 6

This example provides the materials and methods implemented during the execution of Examples 1-5.

### Microscopy

All fluorescence images were taken on a Nikon AXR confocal microscope.

Acquisition parameters were kept consistent within the same experiment and all post-image processing was performed equally across all images of the same experiment. Images were assembled in Adobe Photoshop CC 2023, and Figures were assembled using Adobe Illustrator CC 2023.

### Tissue Processing for Staining and Histology

All tissue or HIOs were placed in 10% Neutral Buffered Formalin (NBF) for 24 hours at room temperature (RT) on a rocker for fixation. Fixed specimens were then washed 3× in UltraPure DNase/RNase-Free Distilled Water (Thermo Fisher Cat #10977015) for 30-60 minutes per wash depending on its size. Next, tissue was dehydrated through a methanol series diluted in UltraPure DNase/RNase-Free Distilled Water for 30-60 minutes per solution: 25% MeOH, 50% MeOH, 75% MeOH, 100% MeOH. Tissue was either immediately processed for paraffin embedding or stored in 100% MeOH at 4° C. for future paraffin processing or whole mount staining. For paraffin processing, dehydrated tissue was placed in 100% EtOH, followed by 70% EtOH, and perfused with paraffin using an automated tissue processor (Leica ASP300) with 1 hour solution changes overnight. Tissue was then placed into tissue cassettes and base molds for sectioning. Prior to sectioning, the microtome and slides were sprayed with RNase Away (Thermo Fisher Cat #700511). 5 μm-thick sections were cut from paraffin blocks onto charged glass slides. Slides were baked for 1 hour at 60° C. in a dry oven and were used within 24 hours for FISH or within a week for IF. Slides were stored at room temperature in a slide box containing a silica desiccator packet and the slide box seams were sealed with parafilm.

### Antibody, Fluorescence In Situ Hybridization (FISH) Probes, and Dye Information

All stains were completed in organoids derived from 3 different stem cell lines, representative images from one line (iPSC72.3) are reported in this manuscript. The following antibodies were used throughout the manuscript in immunofluorescence and fluorescent in situ hybridization with co-immunofluorescence staining. All antibodies were used on FFPE processed sections or whole mount organoids as described below, no frozen sections were used. Rabbit anti-SM22 1:500 (Abcam Cat #abl4106), goat anti-E-Cadherin 1:500 (R&D Systems Cat #AF748), mouse anti-E-Cadherin 1:500 (BD Transduction Laboratories Cat #610181), rabbit anti-PECAM 1:200 (Sigma Cat #HPA004690), mouse anti-TUBB3 1:200 (BioLegend Cat #801201), sheep anti-SM22 1:500 (Novus Cat #AF7886), chicken anti-MAP2 1:2000 (Abcam Cat #ab92434), goat anti-VIM 1:200 (R&D Systems Cat #AF2105), rabbit anti-c-KIT 1:500 (Abcam Cat #ab32363) and mouse anti-VE-CAD 1:1000 (R&D Systems Cat #MAB9381). FISH probes were acquired from ACDbio and stained using the RNAscope multiplex fluorescent manual protocol and kit. RNAscope Probe Hs-EREG (ACD, Cat #313081). Tomato lectin dye was purchased from Thermo Fischer Cat #L32472 and was used 1:1 with sterile PBS for tail vein injections and diluted 1:100 in Tube media (See “RVEC Experiments” section below for Tube Media formulation) for RVEC experiments.

### Immunofluorescence (IF) Protein Staining on 2D Paraffin Sections

Tissue slides were deparaffinized in Histo-Clear II (National Diagnostics Cat #HS-202) twice for 5 minutes each, followed by rehydration through an ethanol series of two washes each for two minutes in 100% EtOH, 95% EtOH, 70% EtOH, 30% EtOH, and finally two washes in ddH2O each for 5 minutes. Antigen retrieval was performed using 1× Sodium Citrate Buffer (100 mM trisodium citrate (Sigma, Cat #S1804), and 0.5% Tween 20 (Thermo Fisher Cat #BP337), pH 6.0). Slides were steamed for 20 minutes then washed three times in ddH2O for 5 minutes each. Slides were then incubated in a humidity chamber at room temperature for 1 hour with blocking solution covering the tissue (5% normal donkey serum (Sigma Cat #D9663) in PBS with 0.1% Tween 20). Slides were then incubated in primary antibody diluted as stated above in blocking solution at 4° C. overnight in a humidity chamber. The next day, slides were washed three times in 1×PBS for 5 minutes each and incubated with secondary antibody (1:500) with DAPI (1:1000) diluted in blocking solution for 1 hour at room temperature in a humidity chamber. Secondary antibodies were raised in donkey and purchased from Jackson Immuno. Slides were then washed 3× in 1×PBS for 5 minutes each and mounted with ProLong Gold (Thermo Fisher Cat #P369300). Immunofluorescent stains were imaged within 2 weeks. Stained slides were stored flat and in the dark at 4° C.

### FISH on 2D Paraffin Sections

FISH staining protocol was performed according to the manufacturer's instructions (ACDbio, RNAscope multiplex fluorescent manual) with a 30 minute protease treatment and a 20 minute antigen retrieval step. IF protein co-stains were added following the ACDBio FISH protocol. Briefly, after blocker is applied to the final channel and washed twice in wash buffer, slides were washed 3× for 5 minutes in PBS followed by the IF protocol stated above from the blocking step onwards. FISH stains were imaged within a week.

Whole Mount IF with Antibody Staining

Organoids were removed from Matrigel using a cut P1000 tip and transferred to a 1.5 mL mini-centrifuge tube. Tubes were spun at 300 g for 5 minutes at 4° C. and supernatant was removed. Organoids were fixed in 10% NBF overnight at room temperature on a rocker. The following day, organoids were washed 3× for 1 hour in organoid wash buffer (OWB) (0.1% Triton, 0.2% BSA in 1×PBS) at room temperature on a rocker. Organoids were then incubated in CUBIC-L (TCI Chemicals Cat #T3740) for 24 hours at 37° C. They were then washed 3× in OWB and permeabilized for 24 hours at 4° C. on a rocker with permeabilization solution (5% normal donkey serum, 0.5% Triton in 1×PBS). After 24 hours of permeabilization, the solution was removed and the desired primary antibody, diluted in OWB, was added. Organoids were incubated overnight at 4° C. on a rocker. The next day, organoids were washed 3× in OWB for 1 hour per wash at room temperature on a shaker. Then, secondary antibody was diluted in OWB at 1:500 and added overnight at 4° C. wrapped in foil on a shaker. Organoids were then washed again the following day 3× in OWB at room temperature with the first wash being for 1 hour with DAPI added at dilution of 1:1,000. The remaining two washes were for 1 hour in OWB only. Organoids were then transferred to a 96-well imaging plate (Thermo Fisher Cat #12-566-70) and cleared using enough CUBIC-R to submerge the organoids (TCI Chemicals Cat #T3741). Organoids remained in CUBIC-R for imaging and whole mount images were imaged within 1 week.

### Stem Cell Lines and Generation of Human Intestinal Organoids (HIOs)

This study includes data from HIOs generated across 3 hPSC lines: Human ES line H9 (NIH registry #0062, RRID: CVCL_9773, female) with an mCherry reporter, human iPSC lines WTC11 (RRID: CVCL_Y803, male) and 72.3³². All experiments using hPSCs were approved by the University of Michigan Human Pluripotent Stem Cell Research Oversight Committee. All stem cell and organoid lines were routinely monitored for mycoplasma using the MycoAlert Mycoplasma Detection Kit (Lonza Cat #LT07-318).

### Stem Cell Maintenance and Differentiation

Maintenance and differentiation into HIOs were carried out as previously described^{1,2,11,16,32,33}. Cells were kept in a 37° C. tissue culture incubator with 5% CO2 and lines were maintained in mTeSR Plus cultured media (Stemcell Technologies Cat #100-1130). Stem cells underwent directed differentiation into definitive endoderm over a 3-day treatment using Activin A (100 ng/mL, R&D Systems Cat #338-AC) added to RPMI base media. This base media was supplemented with 0%, 0.2%, 2% HyClone dFBS (Thermo Fischer Cat #SH3007103) on subsequent days with the addition of 5 mL penicillin-streptomycin each day (Gibco Cat #15070063). After three days, endoderm monolayers were differentiated into an intestinal identity by treatment with FGF4³⁴ (500 ng/mL) and CHIR99021 (2 μM, APExBIO Cat #A8396). On days 4-6 of hindgut differentiation, spheroids budded from the monolayer and were collected. These spheroids were embedded in Matrigel as previously described³² and maintained in basal growth media consisting of Advanced DMEM/F12 (Gibco Cat #11320033) with B27 (50x, Thermo Fisher Cat #17504044), GlutaMAX (1×, Gibco Cat #35050061), penicillin-streptomycin (Gibco Cat #15070063), and HEPES buffer (15 mM, Gibco Cat #15630080). Organoid basal growth media was supplemented with epidermal growth factor (EGF) (100 ng/mL, 10 ng/mL, 1 ng/mL R&D Systems Cat #236-EG-01M) or Epiregulin (EREG) (100 ng/mL, 10 ng/mL, 1 ng/mL R&D Systems Cat #1195-EP-025/CF) with Noggin-Fc (100 ng/mL, purified from conditioned media³⁵), and R-Spondin1 (5% conditioned medium³⁶) for the first three days of culture to pattern a proximal small intestine. On the third day after embedding, media was changed to basal growth media supplemented with EGF or EREG only (no additional Noggin or R-Spondin1) and remained in this media for the duration of the experiments with media changes every 5 days. Organoids were not passaged to avoid disrupting the development and spatial organization of the key cell types seen in EREG-grown HIOs.

### HIO Forming Efficiency Assay

Spheroids were collected from three different stem cell lines for three different batches on days 4-6 of hindgut treatment. Spheroids were plated in Matrigel, counted (day 0), and allowed to grow for 10 days into organoids. After 10 days, organoids were counted and forming efficiency was calculated by taking the number of organoids that had formed at day 10 and dividing it by the total number of spheroids collected on day 0.

### HIO Shape and Area Quantification

To compare shape and area of different HIO conditions, 5 organoids per condition were grown for 30 days in vitro and a 10× bright-field image of each organoid was outlined manually using the freehand selection tool in ImageJ. Outlines were measured in ImageJ with measurements set to capture area and shape descriptors including area, solidity, aspect ratio, circularity, and roundness. This was completed on three stem cell lines and measurements were graphed in FIG. 3.

RNA Extraction, cDNA Synthesis, and RT-qPCR

Three different stem cell lines were used for each experiment with three different organoid differentiations (batches) and three technical replicates for each batch. mRNA was isolated using the MagMAX-96 Total RNA Isolation Kit/machine (Thermo Fisher Cat #AM1830), and RNA quality/yield were then measured using a NanoDrop™ One© spectrophotometer (Thermo Fisher Cat #13-400-519) prior to cDNA synthesis. cDNA synthesis was performed using 100 ng of RNA from each sample leveraging the SuperScript VILO cDNA Kit (Thermo Fisher Cat #11754250). RT-qPCR was performed on a Step One Plus Real-Time PCR System (Thermo Fisher Cat #43765592R) with QuantiTect SYBR Green PCR Kit (QIAGEN Cat #204145). Expression of genes in the measurement of arbitrary units was calculated relative to RN18S using the following equation and reported in bar graphs for each gene analyzed: d2^{RN18S(CT)-GENE(CT)}×1,000.

### Quantification and Statistical Analysis (for RT-qPCR Etc)

All quantitative experiments were completed in 3 different organoid lines for 3 different batches with 3 technical replicates per batch. All statistical analysis was performed in GraphPad Prism Software. See figure legends for number of replicates used, statistical test performed, and the p-values used to determine the significance for each separate analysis. All t tests were ra″n two-tailed, unpaired with welch's correction.

### Mouse Kidney Capsule Transplantation

The University of Michigan and Cincinnati Children's Hospital Institutional Animal Care and Use Committees approved all animal research. HIOs were cultured in vitro for at least 28 days then collected for transplantation. HIOs were implanted under the kidney capsules of immunocompromised NOD-scid IL2Rg-null (NSG) mice^{23,25} (Jackson Laboratory strain no. 0005557). Briefly, mice were anesthetized using 2% isoflurane and a left-flank incision was used to expose the kidney after shaving and sterilization of the area of incision with 3 alternating washes of hibiclens surgical soap and sterile water to prep the area after shaving. Between 1 and 3 HIOs were then surgically implanted beneath mouse kidney capsules using forceps. Prior to closure, an intraperitoneal flush of Zosyn (100 mg kg-1; Pfizer) was administered. Mice were administered a dose of analgesic carprofen during the surgery and an additional dose after 24 hours. All mice were monitored daily for 10 days and then weekly until they were euthanized for retrieval of transplanted HIOs after 10 weeks.

tHIO Vasculature Lectin Labeling

HIOs were transplanted into the kidney capsule of a mouse as described above and allowed to mature for 10 weeks. At 10 weeks, conjugated 647 tomato lectin (Thermo Fischer Cat #L32472) was mixed 1:1 with sterile PBS and 100 μL was drawn into a 30-gauge insulin needle. Mice were given a tail vein injection of the diluted lectin and allowed to move about normally for 5 minutes before they were sacrificed for tHIO harvest. tHIOs were immediately placed in 10% NBF overnight at room temperature on a shaker and the whole mount staining protocol outlined in the previous section was started the following day.

### Flow Cytometry

After lectin injection outlined in the previous section, tHIOs were harvested and minced with dissecting scissors. Tissue was then placed into a 15 mL conical tube containing 9 mL 0.1% (w/v) filter-sterilized Collagenase Type II (Thermo Fisher Cat #17101015) in 1×PBS and 1 mL filter-sterilized 2.5 units/mL dispase II (Thermo Fisher Cat #17105041) in 1×PBS per gram of tissue. The tube was incubated at 37° C. for 30 minutes with mechanical dissociation every 10 minutes. After incubation, 75 μL DNase I was added and incubated at 37° C. for an additional 30 minutes with mechanical dissociation every 10 minutes. Following dissociation, 5 mL of isolation media containing 79% RPMI 1640 (Thermo Fisher Cat #11875093), 20% FBS (Sigma Cat #12103C), and 100 U/mL penicillin-streptomycin (Thermo Fisher Cat #15140122) were added per 10 mL of digestion solution. Cells were filtered through 100 μm and 70 μm filters, pre-coated with isolation media, and centrifuged at 500 g for 5 minutes at 4° C. The cells were washed by adding 2 mL of FACS buffer and centrifuged at 500 g for 5 minutes at 4° C. twice. Cells for all control tubes (unstained, DAPI only, isotype controls, individual antibodies/fluorophores) and experimental cells were placed into a FACS tube for cell sorting (Corning Cat #352063). Cells were stained with primary antibody (CD144) diluted 1:50 in FACS buffer (CD144, VE-Cadherin, anti-human FITC) for 30 minutes at 4° C. Cells were then washed with 5 mL FACS buffer and centrifuging at 500 g for 5 minutes at 4° C. for two washes. Cells were resuspended in FACS buffer and 0.2 μg/mL DAPI was added. FACS was performed using a BD FACS Discovery S8 Cell Sorter and quantitated using the accompanying software.

### Single Cell RNA Sequencing Dissociation

To dissociate HIOs to single cells, organoids were removed from Matrigel using a cut P1000 tip and placed in a 1.5 mL micro-centrifuge tube. All consumables such as tubes and pipette tips used in this prep were pre-washed with 1% BSA in 1×HBSS to prevent adhesion of cells. Following collection, dissociation enzymes and reagents from the Neural Tissue Dissociation Kit (Miltenyi Cat #130-092-628) were used, and all incubation steps were carried out in a refrigerated centrifuge pre-chilled to 10° C. unless otherwise stated. Organoids were treated for 15 minutes at 10° C. with Mix 1 followed by an incubation for 10 min increments at 10° C. with Mix 2. Frequent agitation by pipetting with a P1000 pipette was implemented until organoids were fully dissociated. Cells were passed through a 70 μm filter coated with 1% BSA in 1×HBSS, centrifuged at 500 g for 5 minutes at 10° C. and resuspended in 500 mL 1×HBSS (with Mg2+, Ca2+). Cells were centrifuged 500 g for 5 minutes at 10° C. and washed twice by suspension in 2 mL of HBSS+1% BSA, followed by more centrifugation. Cells were then counted using a hemocytometer, centrifuged and resuspended to reach a concentration of 1000 cells/μL and kept on ice. Single cell libraries were immediately prepared on the 10× Chromium by the University of Michigan Advanced Genomics Core facility with a target capture of 5000 cells. A full, detailed protocol of tissue dissociation for single cell RNA sequencing can be found at www.jasonspencelab.com/protocols.

### Single Nuclei RNA Sequencing Dissociation

Nuclei were isolated and permeabilized in accordance with 10× Genomics' Chromium Nuclei Isolation Kit Protocol (10× Genomics Cat #1000493). Briefly, tissue was minced into smaller fragments and then placed in lysis buffer where it was further dissociated mechanically with a pellet pestle. Tissue was then incubated in the lysis buffer for 5-7 minutes. The suspension was passed through the nuclei isolation column and spun at 16,000 g for 20 seconds at 4° C. The suspension was then vortexed for 10 seconds and centrifuged at 500 g for 3 minutes at 4° C. The supernatant was removed, and the pellet was resuspended in 500 μL of Debris Removal Solution and centrifuged at 700 g for 10 minutes at 4° C. The supernatant was removed, and the pellet was resuspended in 1 mL of Wash Solution and centrifuged at 500 g for 5 minutes at 4° C. twice. The final pellet was resuspended in diluted nuclei buffer. Nuclei capture was carried out on the 10× Chromium platform with a target capture of 5000 nuclei per sample, and libraries were immediately prepared by the University of Michigan Advanced Genomics Core facility.

### Sequencing Library Preparation and Transcriptome Alignment

All single-cell RNA-seq sample libraries were prepared with the 10× Chromium Controller using v3 chemistry (10× Genomics Cat #1000268). Sequencing was performed on a NovaSeq 6000 with targeted depth of 100,000 reads per cell. Default alignment parameters were used to align reads to the pre-prepared human reference genome (hg38) provided by the 10× Cell Ranger pipeline. Initial cell demultiplexing and gene quantification were also performed using the default 10× Cell Ranger pipeline.

### Sequencing Data Analysis

To generate cell-by-gene matrices, raw data was processed using the 1OX Cell Ranger package, and sequenced reads were aligned to the human genome hg38. All downstream analysis was carried out using Scanpy³⁷ or Seurat²⁷ (depending on package usage needs). For primary human tissue sample analysis in FIG. 1, the human whole cell fetal dataset previously published^{10,19,26} was reanalyzed. Samples included a 47-day proximal intestine, a 59-day proximal intestine, two 72-day duodenum, 80-day duodenum and ileum, an 85-day duodenum, 101-day duodenum and ileum, two 127-day duodenums, 132-day duodenum. All samples were filtered to remove cells with less than 500 or greater than 10,000 genes, or greater than 60,000 unique molecular identifier (UMI) counts per cell. De-noised data matrix read counts per gene were log normalized prior to analysis. After log normalization, highly variable genes were identified and extracted, and batch correction was performed using the BBKNN algorithm. The normalized expression levels then underwent linear regression to remove effects of total reads per cell and cell cycle genes, followed by a z-transformation. Dimension reduction was performed using principal component analysis (PCA) and then uniform manifold approximation and projection (UMAP) on the top 16 principal components (PCs) and 30 nearest neighbors for visualization on 2 dimensions. Clusters of cells within the data were calculated using the Louvain algorithm within Scanpy with a resolution of 1.09. Cell lineages were identified using canonically expressed genes covering 47,100 intestinal cells from all samples.

For FIG. 4, all organoid whole cell samples (1 ng/ml EREG, 10 ng/ml EREG, 100 ng/ml EREG, 100 ng/ml EGF) were filtered to remove cells with less than 700 or greater than 6,800 genes, or greater than 33,000 UMI counts per cell, and 0.1 mitochondrial cell counts. Data matrix read counts per gene were log normalized prior to analysis. After log normalization, highly variable genes were identified and extracted, no batch correction was needed as these samples were processed at the same time. Data was then scaled by z-transformation. Dimension reduction was performed using PCA and then UMAP on the top 10 PCs and 15 nearest neighbors for visualization. Clusters of cells within the data were calculated using the Louvain algorithm within Scanpy with a resolution of 0.4. The 10 ng/mL EREG sample alone was filtered to remove cells with less than 700 or greater than 8,000 genes, or greater than 50,000 UMI counts per cell, and 0.1 mitochondrial cell counts. Data matrix read counts per gene were log normalized prior to analysis. After log normalization, highly variable genes were identified and extracted. Data was then scaled by z-transformation. Dimension reduction was performed using PCA and then UMAP on the top 10 PCs and 15 nearest neighbors for visualization. Clusters of cells within the data were calculated using the Louvain algorithm within Scanpy with a resolution of 0.4. The 1 ng/mL EREG sample alone was filtered to remove cells with less than 500 or greater than 8,000 genes, or greater than 45,000 UMI counts per cell, and 0.1 mitochondrial cell counts. Data matrix read counts per gene were log normalized prior to analysis. After log normalization, highly variable genes were identified and extracted. Data was then scaled by z-transformation. Dimension reduction was performed using PCA and then UMAP on the top 10 PCs and 15 nearest neighbors for visualization. Clusters of cells within the data were calculated using the Louvain algorithm within Scanpy with a resolution of 0.4.

For FIG. 1, 10 ng/ml EREG single nuclei dataset from in vitro grown HIOs was first processed through the standard CellBender³⁸ workflow to remove ambient RNA introduced in the nuclei isolation preparation. Then the same dataset was filtered to remove cells with less than 1200 or greater than 6,000 genes, or greater than 17,500 UMI counts per cell, and 0.1 mitochondrial cell counts. Data matrix read counts per gene were log normalized prior to analysis. After log normalization, highly variable genes were identified and extracted. Data was then scaled by z-transformation. Dimension reduction was performed using PCA and then UMAP on the top 18 PCs and 15 nearest neighbors for visualization. Clusters of cells within the data were calculated using the Louvain algorithm within Scanpy with a resolution of 0.5. For FIG. 2, the 10 ng/mL EREG single nuclei dataset from transplanted HIOs and was first processed through the standard CellBender³⁸ workflow to remove ambient RNA introduced in the nuclei isolation preparation. Then the same dataset was filtered to remove cells with less than 400 or greater than 8,000 genes, or greater than 30,000 UMI counts per cell, and 0.2 mitochondrial cell counts. Data matrix read counts per gene were log normalized prior to analysis. After log normalization, highly variable genes were identified and extracted. Data was then scaled by z-transformation. Dimension reduction was performed using PCA and then UMAP on the top 15 PCs and 15 nearest neighbors for visualization. Clusters of cells within the data were calculated using the Louvain algorithm within Scanpy with a resolution of 0.5.

Label Transfer of tHIO Dataset onto Human Fetal Dataset

We utilized Seurat's recommended pipeline to perform single-cell reference mapping using the same cells as the reference data (human fetal intestine) and query data (tHIOs). PCAs are first performed on reference and query data. Then a set of anchors are identified and filtered based on the default setting of the function FindTransferAnchors. With the computed anchors, reference.reduction parameter set to PCA, and reduction.model set to UMAP, the function MapQuery returns the projected UMAP coordinates of the query cells mapped onto the reference UMAP. The projected UMAP (colored in red) and the reference UMAP (colored in light gray) were reintegrated to visualize the result of the reference-based mapping in FIG. 7.

### Endoderm Atlas

Reference map embedding to the Human Fetal Endoderm Atlas¹⁹ to determine off target lineages was performed using the scoreHIO R Package. tHIO samples were processed following the preprocessing steps outlined above in the Single-cell data analysis section and then put through the basic workflow outlined for this package to map tHIO cells onto the reference endodermal organ atlas.

### Cell Scoring Analysis

Cells were scored based on expression of the 20 most differentially expressed genes per tissue type in the human fetal reference dataset. See supplement for gene lists. After obtaining the log-normalized and scaled expression values for the data set, scores for each cell were calculated as the average z score within each set of selected genes. tHIO muscle contractions and ENS function Muscle contraction and ENS function was assayed as previously described^{12,39}.

Following HIO transplantation as outlined in the previous section, tHIOs were matured for 10-12 weeks before harvest. tHIOs were cut into strips ~2×6 mm in size and the epithelium mechanically removed as previously described¹². No chelation buffer was used, and all manipulations occurred in oxygenated Kreb's buffer while on ice ((NaCl, 117 mM; KCl, 4.7 mM; MgCl2, 1.2 mM; NaH2PO4, 1.2 mM; NaHCO₃, 25 mM; CaCl2), 2.5 mM and glucose, 11 mM), warmed at 37° C. and gassed with 95% 02+5% CO2). These strips were mounted in an organ bath chamber system (Radnoti) to isometric force transducers (ADInstruments) and contractile activity was continuously monitored and recorded using LabChart software (ADInstruments). All measurements were normalized to muscle strip mass. After an equilibrium period, a logarithmic dose response to Bethanechol (Sigma-Aldrich Cat #C5259) was obtained through the administration of exponential doses with concentrations of 1 nM to 10 mM at 2 min intervals before the administration of 10 μM scopolamine (Tocris Bioscience Cat #1414/1G). After another equilibrium period, tissue strips were then stimulated with dimethyl phenyl piperazinium (DMPP) (10 μM, Sigma, Cat #D5891). NG-nitro-L-arginine methyl ester (L-NAME) (50 μM, Sigma Cat #N5751) was added 10 minutes before DMPP stimulation to observe the effects of NOS inhibition. Without washing, atropine sulfate salt monohydrate (Atropine) (1 μM, Sigma Cat #A0132) was then applied 10 minutes prior to a final DMPP stimulation to observe the cumulative effect of NOS and Ach receptor inhibition. After several washes and an additional equilibrium period, another dose of DMPP was administered. Neurotoxin tetrodotoxin (TTX) (4 μM, Tocris Cat #1078) was added 5 minutes before a final DMPP stimulation and measurement. Analysis was performed by calculating the integral (expressed as area under the curve, AUC) immediately before and after stimulation for 60 seconds.

### RVEC Experiments—Culturing and Maintenance

RVECs were obtained as previously described²⁹. Briefly, various multiplicity of infection (MOI) (from 5 to 20) of lentiviral vectors expressing the transcription factor ETV2 was transduced into human umbilical vein endothelial cells (HUVECs) to generate R-VECs. Then the transduced ECs that generated the most functional perfusable and durable vascular network on the microfluidic devices were selected for further experimentation. The following protocol was implemented to propagate these cells: RVECs were grown in T75 flasks coated in 0.2% gelatin in Endothelial Cell (EC) medium which is comprised of 400 ml M199 (Gibco Cat #11150067), 100 ml HyClone dFBS (Fisher Cat #SH3007103), 5 mL GlutaMAX (1×, Gibco Cat #35050061), 5 mL penicillin-streptomycin (Gibco Cat #15070063), 7.5 mL HEPES buffer (15 mM, Gibco Cat #15630080), Heparin (Sigma Cat #H3149-100KU), FGF2 (10 ng/mL, R&D Cat #233-FB-MTO), IGF1 (10 ng/mL, Preprotech Cat #100-11), EGF (10 ng/mL, R&D Systems Cat #236-EG-01M) and N-acetylcysteine (1.5 mM, Sigma Cat #A9165-25G). The cells were split 1:3 using Accutase (Corning Cat #MT25058CI) and passaged on gelatin coated flasks.

### Lentiviral Labeling

RVECs were transduced with GFP lenti-particles (Lenti-EV-GFP-VSVG) provided by the University of Michigan Vector Core. Virus was diluted in EC media and added to the RVECs for 8 hours. After RVECs were incubated with the virus, the cells were thoroughly washed and allowed to continue to grow normally.

### Microfluidic Device

Polydimethylsiloxane (PDMS; Sylgard 184; Ellsworth Adhesives Cat #2065622) based microfluidic devices were fabricated via soft lithography with a 3D printed resin cast. The device is 50 mm in length, 20.64 mm in width, and 3 mm in height. The physical chamber housing the 3D co-culture carries a height of 1.5 mm to account for larger size HIOs. Each device was plasma treated, with Harricks Expanded Plasma Cleaner (Harricks Plasma Cat #PDC-001), to a 24×60 mm glass cover slip (VWR Cat #152460), and then placed in an 80° C. oven for at least 1 hour to finalize a strong adhesion. For long term storage, devices were sealed with parafilm. Before use, devices were sterilized with UV light for at least 30 minutes prior to seeding of cells.

RVECs were washed with sterile PBS then incubated in accutase for 3-5 minutes. Digestion was stopped by adding an equal volume of EC media and cell suspension was obtained by centrifugation at 500 g for 5 minutes at 4° C. Supernatant was removed and RVECs were resuspended in M199 (Gibco Cat #11150067) and counted. 250,000 RVEC was aliquoted into a 1.5 mL micro-centrifuge tube, which corresponds to a single lane on the microfluidic device.

HIOs were removed from Matrigel using a cut P200 tip and transferred to a 1.5 mL micro-centrifuge tube to be spun at 300 g for 5 minutes at 4° C. The supernatant was removed and resuspended in DMEM. Three to five HIOs were then picked and added to each aliquot of RVECs in the 1.5 mL micro-centrifuge tube, which was subsequently centrifuged at 500 g for 5 minutes at 4° C. Supernatant was removed and resuspended in 32 μL of a Fibrin mixture consisting of Fibrinogen from bovine plasma (Sigma Cat #F8630), Human Fibrinogen 1 Plasminogen Depleted (Enzyme Research Lab Cat #FIB-1), and X-Vivo 20 (Lonza Cat #190995). 3.6 uL of a Thrombin mixture, consisting of Thrombin from bovine plasma (Sigma Cat #T4648) and X-Vivo 20, was then added to the mixture of RVECs and Fibrinogen. The final matrix concentration is 0.5 mg/mL of Human Fibrinogen, 2 mg/mL of Bovine Fibrinogen, and 2 U/mL of Thrombin. The cell mixture was resuspended and immediately seeded into the microfluidic chamber within 10-15 seconds. Between loading each lane, devices were flipped upside down to prevent HIOs resting to the bottom. Devices were then incubated, right side up, for 5-15 minutes.

40 μL of Tube Media (500 mL StemSpan SFEM, Stemcell Technologies Cat #9650; 50 mL Knockout Serum, Gibco Cat #10828010; 5 mL Penicillin-Streptomycin, Gibco Cat #15070063; 5 mL Heparin, Sigma Cat #H3149; 5 mL GlutaMAX, 1×Gibco Cat #15070063; 5 mL HEPES Buffer, Gibco Cat #15630080; 10 ng/mL FGF2, R&D Cat #233-FB-MTO; 10 ng/mL Aprotinin, Sigma Cat #A6106) was added to both inlet and outlet. A 1 mL syringe, without the plunger, was additionally attached to both ends and 1 mL of Tube media was added to the inlet syringe to induce shear stress via gravity. Media in the outlet was recycled back to the inlet on a daily basis.

### Data and Code Availability Statement

Sequencing data generated and used by this study are deposited at EMBL-EBI ArrayExpress. Data sets for human fetal intestine (ArrayExpress: E-MTAB-9489, www.ebi.ac.uk/arrayexpress/experiments/E-MTAB-9489/, and previously published work²⁶); Data sets for whole cell single cell sequencing of 1 ng/mL, 10 ng/mL, 100 ng/mL EREG HIOs and 100 ng/mL EGF HIO (ArrayExpress: E-MTAB-13463, www.ebi.ac.uk/arrayexpress/experiments/E-MTAB-13463/,); Data sets for single nuclear RNA sequencing of 10 ng/mL HIOs and 10 ng/mL EREG tHIOs (ArrayExpress: E-MTAB-13469, www.ebi.ac.uk/arrayexpress/experiments/E-MTAB-13469/). Code used to process raw data can be found at github.com/jason-spence-lab/Childs_2023.git

### Example 7

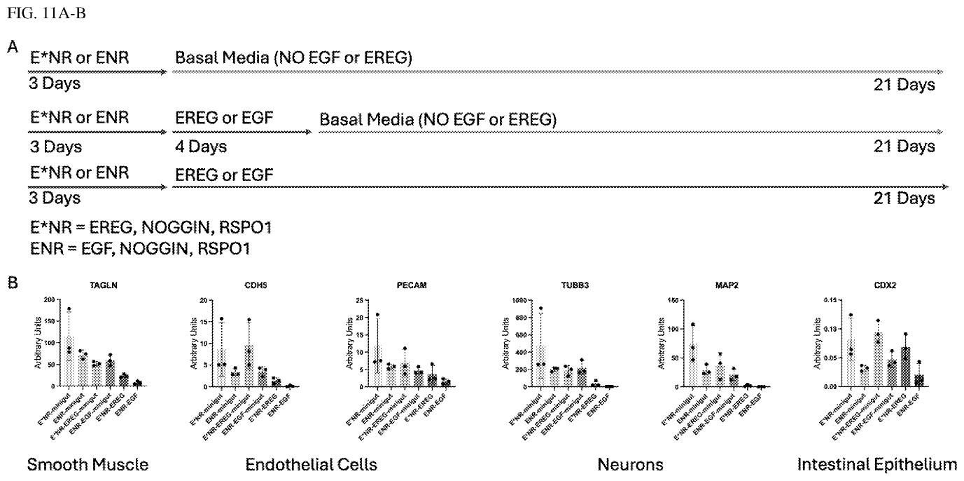

This example demonstrates that when human intestinal organoids are grown in EGF for any length of time, the observed cell lineages in EREG-grown organoids (smooth muscle, neurons, and endothelial cells) differ significantly. FIGS. 11A and 11B demonstrate that human intestinal organoids grown in EREG consistently show higher expression of genes associated with these cell types compared to their EGF counterparts. These results indicate that EREG is uniquely patterning these cell types, whereas EGF either does not promote their formation or actively inhibits it.

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### INCORPORATION BY REFERENCE

The entire disclosure of each of the patent documents and scientific articles referred to herein is incorporated by reference for all purposes.

The following references denoted throughout this application are incorporated by reference in their entireties:
1. Spence J R, Mayhew C N, Rankin S A, et al. Directed differentiation of human pluripotent stem cells into intestinal tissue in vitro. Nature. 2011; 470(7332):105-109. doi:10.1038/nature09691
2. Tsai Y H, Nattiv R, Dedhia P H, et al. In vitro patterning of pluripotent stem cell-derived intestine recapitulates in vivo human development. Development. 2017; 144(6):1045-1055. doi:10.1242/DEV.138453
3. Krishnamurthy M, Kechele D O, Broda T, et al. Using Human Induced Pluripotent Stem Cell-Derived Organoids to Identify New Pathologies in Patients With PDX1 Mutations. Gastroenterology. 2022; 163(4):1053-1063.e7. doi:10.1053/J.GASTRO.2022.06.083
4. McCauley H A, Matthis A L, Enriquez J R, et al. Enteroendocrine cells couple nutrient sensing to nutrient absorption by regulating ion transport. Nat Commun. 2020; 11(1). doi:10.1038/S41467-020-18536-Z
5. Sarvestani S K, Signs S, Hu B, et al. Induced organoids derived from patients with ulcerative colitis recapitulate colitic reactivity. Nat Commun. 2021; 12(1). doi:10.1038/S41467-020-20351-5
6. Finkbeiner S R, Hill D R, Altheim C H, et al. Transcriptome-wide Analysis Reveals Hallmarks of Human Intestine Development and Maturation In Vitro and In Vivo. Stem Cell Reports. 2015; 4(6):1140-1155. doi:10.1016/j.stemcr.2015.04.010
7. Childs C J, Eiken M K, Spence J R. Approaches to benchmark and characterize in vitro human model systems. Development. 2022; 149(20). doi:10.1242/DEV.200641/277169
8. Cortez A R, Poling H M, Brown N E, Singh A, Mahe M M, Helmrath M A. Transplantation of human intestinal organoids into the mouse mesentery: A more physiologic and anatomic engraftment site. Surgery. 2018; 164(4):643-650. doi:10.1016/J.SURG.2018.04.048
9. Watson C L, Mahe M M, Mdnera J, et al. An in vivo model of human small intestine using pluripotent stem cells. Nat Med. 2014; 20(11):1310-1314. doi:10.1038/nm.3737
10. Capeling M M, Huang S, Childs C J, et al. Suspension culture promotes serosal mesothelial development in human intestinal organoids. Cell Rep. 2022; 38(7):110379. doi:10.1016/J.CELREP.2022.110379
11. Holloway E M, Wu J H, Czerwinski M, Capeling M M, Glass I, Correspondence JRS. Differentiation of Human Intestinal Organoids with Endogenous Vascular Endothelial Cells. Dev Cell. 2020; 54:516-528.e7. doi:10.1016/j.devcel.2020.07.023
12. Workman M J, Mahe M M, Trisno S, et al. Engineered human pluripotent-stem-cell-derived intestinal tissues with a functional enteric nervous system. Nat Med. 2017; 23(1):49-59. doi:10.1038/N M.4233
13. Holloway E M, Capeling M M, Spence J R. Biologically inspired approaches to enhance human organoid complexity. Development. 2019; 146(8). doi:10.1242/DEV.166173
14. Schlieve C R, Fowler K L, Thornton M, et al. Neural Crest Cell Implantation Restores Enteric Nervous System Function and Alters the Gastrointestinal Transcriptome in Human Tissue-Engineered Small Intestine. Stem Cell Reports. 2017; 9(3):883-896. doi:10.1016/J.STEMCR.2017.07.017
15. Childs C J, Holloway E M, Sweet C W, et al. EPIREGULIN creates a developmental niche for spatially organized human intestinal enteroids. JCI Insight. 2023; 8(6). doi:10.1172/JCI.INSIGHT.165566
16. McCracken K W, Howell J C, Wells J M, Spence J R. Generating human intestinal tissue from pluripotent stem cells in vitro. Nat Protoc. 2011; 6(12):1920-1928. doi:10.1038/nprot.2011.410
17. Múnera J O, Sundaram N, Rankin S A, et al. Differentiation of Human Pluripotent Stem Cells into Colonic Organoids via Transient Activation of BMP Signaling. Cell Stem Cell. 2017; 21(1):51-64.e6. doi:10.1016/j.stem.2017.05.020
18. Yu Q, Kilik U, Holloway E M, et al. Charting human development using a multi-endodermal organ atlas and organoid models In brief 11 Charting human development using a multi-endodermal organ atlas and organoid models. Cell. 2021; 184:3281-3298. doi:10.1016/j.cell.2021.04.028
19. Yu Q, Kilik U, Holloway E M, et al. Charting human development using a multi-endodermal organ atlas and organoid models. Cell. 2021; 184(12):3281-3298.e22. doi:10.1016/j.cell.2021.04.028
20. Finkbeiner S R, Hill D R, Altheim C H, et al. Transcriptome-wide Analysis Reveals Hallmarks of Human Intestine Development and Maturation In Vitro and In Vivo. Stem Cell Reports. 2015; 4(6):1140-1155. doi:10.1016/J.STEMCR.2015.04.010
21. Koenitzer J R, Wu H, Atkinson J J, Brody S L, Humphreys B D. Single-nucleus RNA-sequencing profiling of mouse lung reduced dissociation bias and improved rare cell-type detection compared with single-cell RNA sequencing. Am J Respir Cell Mol Biol. 2020; 63(6):739-747. doi:10.1165/RCMB.2020-0095MA/SUPPL_FILE/DISCLOSURES.PDF
22. Tasic B, Yao Z, Graybuck L T, et al. Shared and distinct transcriptomic cell types across neocortical areas. Nature. 2018; 563(7729):72-78. doi:10.1038/S41586-018-0654-5
23. Darmanis S, Sloan S A, Zhang Y, et al. A survey of human brain transcriptome diversity at the single cell level. Proc Natl Acad Sci USA. 2015; 112(23):7285-7290. doi:10.1073/PNAS.1507125112/-/DCSUPPLEMENTAL
24. Singh A, Poling H M, Chaturvedi P, et al. Transplanted human intestinal organoids: a resource for modeling human intestinal development. Dev. 2023; 150(9). doi:10.1242/DEV.201416/2792717/DEV201416.PDF 25. Watson C L, Mahe M M, Mdnera J, et al. An in vivo model of human small intestine using pluripotent stem cells. Nat Med 2014 2011. 2014; 20(11):1310-1314. doi:10.1038/nm.3737
26. Holloway E M, Czerwinski M, Tsai Y H, et al. Mapping Development of the Human Intestinal Niche at Single-Cell Resolution. Cell Stem Cell. 2020; 28(3). doi:10.1016/j.stem.2020.11.008
27. Hao Y, Hao S, Andersen-Nissen E, et al. Integrated analysis of multimodal single-cell data. Cell. 2021; 184(13):3573-3587.e29. doi:10.1016/J.CELL.2021.04.048
28. Eicher A K, Kechele D O, Sundaram N, et al. Functional human gastrointestinal organoids can be engineered from three primary germ layers derived separately from pluripotent stem cells. Cell Stem Cell. 2022; 29(1):36-51.e6. doi:10.1016/J.STEM.2021.10.010
29. Sanders K M, Koh S D, Ward S M. INTERSTITIAL CELLS OF CAJAL AS PACEMAKERS IN THE GASTROINTESTINAL TRACT. https://doi.org/101146/annurev.physiol68040504094718. 2006; 68(18):307-343. doi:10.1146/ANNUREV.PHYSIOL.68.040504.094718
30. Palikuqi B, Nguyen D H T, Li G, et al. Adaptable haemodynamic endothelial cells for organogenesis and tumorigenesis. Nat 2020 5857825. 2020; 585(7825):426-432. doi:10.1038/s41586-020-2712-z
31. Robertson R T, Levine S T, Haynes S M, et al. Use of labeled tomato lectin for imaging vasculature structures. Histochem Cell Biol. 2015; 143(2):225-234. doi:10.1007/S00418-014-1301-3/FIGURES/2
32. McCracken K W, Cati E M, Crawford C M, et al. Modelling human development and disease in pluripotent stem-cell-derived gastric organoids. Nat 2014 5167531. 2014; 516(7531):400-404. doi:10.1038/naturel3863

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A composition comprising human intestinal organoid tissue derived from human pluripotent stem cells, wherein the human intestinal organoid tissue comprises epithelial cells, stromal cells, neural cells, endothelial cells, smooth muscle cells, and interstitial cells of Cajal (ICCs).

2. The composition of claim 1, wherein the human intestinal organoid tissue culture medium comprises added epiregulin (EREG).

3. The composition of claim 2, wherein the added EREG concentration in the medium is at least 1 ng/ml.

4. The composition of claim 1, wherein the neural cells comprise neuronal and glial cells.

5. The composition of claim 1, wherein the epithelial cells express CDX2, the neural cells express TUBB3 and/or MAP2, the endothelial cells express PECAM and/or CDH5, and the smooth muscle cells express SM22 and/or TAGLN at the protein and/or RNA level.

6. The composition of claim 1, wherein the human intestinal organoid tissue has been maintained and/or expanded for at least 10 hours.

7. The composition of claim 1, wherein the human pluripotent stem cells are human induced pluripotent stem cells or human embryonic stem cells.

8. The composition of claim 1, wherein the human intestinal organoid tissue is genetically engineered.
